SEQUENCE LISTING (8 sequences)

SEQ ID NO: 1 - DNA, 11856 bp, Artificial sequence
  Plasmid
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcgctgaggatgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctagttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcagtaaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctccgcggtgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 2 - DNA, 15539 bp, Artificial sequence
  Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggcgcgccactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagctgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtatgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaatgttgtcctggttgctgtggttaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaaccacccgggccccccctcgaggtcgacggtatcgataagcttgatatcgaattcctgcagcccgggggatccactagttctagagcggccgctctagaactagtaccacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttactcttaattaatcaaaaagttaaaattgtacgaatagattcaccacttcttaacaaatcaaacccttcattgattttctcgaatggcaatacatgtgtaattaaaggatcaagagcaaacttcttcgccataaagtcggcaacaagttttggaacactatccttgctcttaaaaccgccaaatatagctcccttccatgtacgaccgcttagcaacagcataggattcatcgacaaattttgtgaatcaggaggaacacctacgatcacactgactccatatgcctcttgacagcaggacaacgcagttaccatagtatcaagacggcctataacttcaaaagagaaatcaactccaccgtttgacatttcagtaaggacttcttgtattggtttcttataatcttgagggttaacacattcagtagccccgacctccttagcttttgcaaatttgtccttattgatgtctacacctataatcctcgctgcgcctgcagctttacaccccataataacgcttagtcctactcctcctaaaccgaatactgcacaagtcgaaccctgtgtaacctttgcaactttaactgcggaaccgtaaccggtggaaaatccgcaccctatcaagcaaactttttccagtggtgaagctgcatcgattttagcgacagatatctcgtccaccactgtgtattgggaaaatgtagaagtaccaaggaaatggtgtataggtttccctctgcatgtaaatctgcttgtaccatcctgcatagtacctctaggcatagacaaatcatttttaaggcagaaattaccctcaggatgtttgcagactctacacttaccacattgaggagtgaacagtgggatcactttatcaccaggacgaacagtggtaacaccttcacctatggattcaacgattccggcagcctcgtgtcccgcgattactggcaaaggagtaactagagtgccactcaccacatggtcgtcggatctacagattccggtggcaaccatcttgattctaacctcgtgtgcttttggtggcgctacttctacttcttctatgctaaacggctttttctcttcccacaaaactgccgctttacacttaataactttaccggctgttgacatcctcagctagctattgtaatatgtgtgtttgtttggattattaagaagaataattacaaaaaaaattacaaaggaaggtaattacaacagaattaagaaaggacaagaaggaggaagagaatcagttcattatttcttctttgttatataacaaacccaagtagcgatttggccatacattaaaagttgagaaccaccctccctggcaacagccacaactcgttaccattgttcatcacgatcatgaaactcgctgtcagctgaaatttcacctcagtggatctctctttttattcttcatcgttccactaacctttttccatcagctggcagggaacggaaagtggaatcccatttagcgagcttcctcttttcttcaagaaaagacgaagcttgtgtgtgggtgcgcgcgctagtatctttccacattaagaaatataccataaaggttacttagacatcactatggctatatatatatatatatatatatgtaacttagcaccatcgcgcgtgcatcactgcatgtgttaaccgaaaagtttggcgaacacttcaccgacacggtcatttagatctgtcgtctgcattgcacgtcccttagccttaaatcctaggcgggagcattctcgtgtaattgtgcagcctgcgtagcaactcaacatagcgtagtctacccagtttttcaagggtttatcgttagaagattctcccttttcttcctgctcacaaatcttaaagtcatacattgcacgactaaatgcaagcatgcggatcccccgggctgcaggaattcgatatcaagcttatcgataccgtcgactggccattaatctttcccatattagatttcgccaagccatgaaagttcaagaaaggtctttagacgaattacccttcatttctcaaactggcgtcaagggatcctggtatggttttatcgttttatttctggttcttatagcatcgttttggacttctctgttcccattaggcggttcaggagccagcgcagaatcattctttgaaggatacttatcctttccaattttgattgtctgttacgttggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacggccctgcaggcctcagctcttgttttgttctgcaaataacttacccatctttttcaaaactttaggtgcaccctcctttgctagaataagttctatccaatacatcctatttggatctgcttgagcttctttcatcacggatacgaattcattttctgttctcacaattttggacacaactctgtcttccgttgccccgaaactttctggcagttttgagtaattccacataggaatgtcattataactctggttcggaccatgaatttccctctcaaccgtgtaaccatcgttattaatgataaagcagattgggtttatcttctctctaatggctagtcctaattcttggacagtcagttgcaatgatccatctccgataaacaataaatgtctagattctttatctgcaatttggctgcctagagctgcggggaaagtgtatcctatagatccccacaagggttgaccaataaaatgtgatttcgatttcagaaatatagatgaggcaccgaagaaagaagtgccttgttcagccacgatcgtctcattactttgggtcaaattttcgacagcttgccacagtctatcttgtgacaacagcgcgttagaaggtacaaaatcttcttgctttttatctatgtacttgcctttatattcaatttcggacaagtcaagaagagatgatatcagggattcgaagtcgaaattttggattctttcgttgaaaattttaccttcatcgatattcaaggaaatcattttattttcattaagatggtgagtaaatgcacccgtactagaatcggtaagctttacacccaacataagaataaaatcagcagattccacaaattccttcaagtttggctctgacagagtaccgttgtaaatccccaaaaatgagggcaatgcttcatcaacagatgatttaccaaagttcaaagtagtaataggtaacttagtctttgaaataaactgagtaacagtcttctctaggccgaacgatataatttcatggcctgtgattacaattggtttcttggcattcttcagactttcctgtattttgttcagaatctcttgatcagatgtattcgacgtggaattttccttcttaagaggcaaggatggtttttcagccttagcggcagctacatctacaggtaaattgatgtaaaccggctttctttcctttagtaaggcagacaacactctatcaatttcaacagttgcattctcggctgtcaataaagtcctggcagcagtaaccggttcgtgcatcttcataaagtgcttgaaatcaccatcagccaacgtatggtgaacaaacttaccttcgttctgcactttcgaggtaggagatcccacgatctcaacaacaggcaggttctcagcataggagcccgctaagccattaactgcggataattcgccaacaccaaatgtagtcaagaatgccgcagcctttttcgttcttgcgtacccgtcggccatataggaggcatttaactcattagcatttcccacccatttcatatctttgtgtgaaataatttgatctagaaattgcaaattgtagtcacctggtactccgaatatttcttctatacctaattcgtgtaatctgtccaacagatagtcacctactgtatacattttgtttactagtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgaaaaagcgtgttttttattcaaaatgattctaactcccttacgtaatcaaggaatctttttgccttggcctccgcgtcattaaacttcttgttgttgacgctaacattcaacgctagtatatattcgtttttttcaggtaagttcttttcaacgggtcttactgatgaggcagtcgcgtctgaacctgttaagaggtcaaatatgtcttcttgaccgtacgtgtcttgcatgttattagctttgggaatttgcatcaagtcataggaaaatttaaatcttggctctcttgggctcaaggtgacaaggtcctcgaaaatagggcgcgccccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 3 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 4 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAELRQAMPFTQSGDDDAFKIYQ

SEQ ID NO: 5 - protein, 571 aa, Streptococcus mutans
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 6 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 7 - protein, 375 aa, Equus caballus
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 8 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

(19) United States
(12) Patent Application Publication
(10) Pub. No.: US 2013/0040340 A1
(43) Pub. Date: Feb. 14, 2013

(54) PRODUCTION OF ALCOHOL ESTERS IN SITU USING ALCOHOLS AND FATTY ACIDS PRODUCED BY MICROORGANISMS

(71) Applicants: Michael Dauner, Wilmington, DE (US); Robert Dicosimo, Chadds Ford, PA (US); Ranjan Patnaik, Newark, DE (US)

(72) Inventors: Michael Dauner, Wilmington, DE (US); Robert Dicosimo, Chadds Ford, PA (US); Ranjan Patnaik, Newark, DE (US)

(73) Assignee: E. I. DU PONT DE NEMOURS AND COMPANY, Wilmington, DE (US)

(21) Appl. No.: 13/366,511

(22) Filed: Feb. 6, 2012

Related U.S. Application Data
(60) Provisional application No. 61/440,037, filed on Feb. 7, 2011.

(51) Int. Cl. C12P 7/64; C12N 9/20; C12P 39/00

(52) U.S. Cl. 435/42; 435/134; 435/198

(57) ABSTRACT

Fatty acid alcohol esters that may be used as biodiesel are synthesized from microbially produced alcohol and fatty acids using an in situ process. The process utilizes a catalyst capable of esterifying the alcohol and fatty acid products of microorganisms, which are produced from renewable resources.

### FIELD OF THE INVENTION

The invention relates to the fields of microbiology, fermentation, and biofuel production. More specifically, methods are provided for making in situ alcohol esters from microbially produced alcohols and microbially produced fatty acids.

### BACKGROUND OF THE INVENTION

Replacement of petroleum products with renewable, environmentally friendly products is desired to provide a continued resource and benefit the environment by reducing carbon dioxide release to the atmosphere. Fossil fuels may be replaced with or blended with biodiesel fuel for use in diesel engines to decrease the demand for petroleum oil. Biodiesel fuel may also be used as a heating fuel. Biodiesel refers to fatty acid alcohol esters that are typically made by reacting lipids of vegetable oil or animal fat with an alcohol, typically methanol. Fatty acid alcohol esters have many other commercial uses including in cosmetic and food industries as emulsifiers, emollients, and thickeners, as nonionic surfactants in personal care and household products such as detergents, and in waxes, gums, resins, pharmaceutical salves and lotions, lubricating oil additives, finishing agents, and solvents.

Microorganisms can convert carbon sources obtained from renewable feedstocks, or biomass, to products as biocatalysts in fermentation. Various microorganisms produce alcohol and fatty acid products naturally such as acetone, butanol and ethanol production by Clostridia (Acetone—Butanol—Ethanol (ABE) fermentation), ethanol production by yeast and Zymomonas, and oil production by oleaginous yeast, protists and microalgae. In addition, specific microorganisms have been genetically engineered for production of alcohols to be used in commercial processes. For example, E. coli has been engineered for production of 1-butanol, 1-propanol, and isopropanol (Shen and Liao (2008) Metab. Eng. 10(6):312-320; Hanai et al. (2007) Appl. Environ. Microbiol. 73(24):7814-7818; US20080293125), Saccharomyces cerevisiae has been engineered for production of butanol (U.S. Pat. No. 7,851,188), and Zymomonas mobilis has been engineered as a production strain for ethanol (U.S. Pat. No. 7,741,119). In addition, microorganisms have been engineered for increased commercial production of fatty acids. For example, E. coli has been engineered for increased production of fatty acids (Lu et al. (2008) Metabolic Eng. 10:333-339; Lennen et al. (2010) Biotech. and Bioeng. 106:193-202) and Yarrowia lipolytica has been engineered for production of high levels of eicosapentaenoic acid (cis-5, 8, 11, 14, 17-eicosapentaenoic acid; ω-3; U.S. Pat. Appl. Pub. 2006-0115881A1).

Biodiesel is typically made by reacting oils and alcohols in a base catalyzed chemical reaction resulting in transesterification. Acid catalysis is also used to esterify fatty acids with alcohol. Typically vegetable oil or animal fat is used as a source of fatty acids, and the fatty acids are generally in the form of triacylglycerols (esters containing three fatty acids and the trihydric alcohol, glycerol). In a base catalyzed reaction of triacylglycerols in the presence of excess alcohol, a transesterification reaction produces glycerol and alcohol esters. Typically methanol or ethanol is the alcohol used and fatty acid methyl esters (FAME) or fatty acid ethyl esters (FAEE) are produced as biodiesel.

Fatty acid alcohol esters have also been produced intracellularly by engineered E. coli producing ethanol using a pathway from Zymomonas and expressing a wax ester synthase/acyl-coenzyme A:diacylglycerol aceltransferase (WS/DGAT) from Acinetobacter baylyi (Kalscheuer et al. (2006) Microbiol. 152:2529-2536). This enzyme uses fatty acid-coenzyme A thioesters as substrates for esterification with alcohols or glycerol.

There is a need for alternative effective processes for production of fatty acid alcohol esters for biodiesel and other applications, using renewably sourced fatty acids and alcohols.

### SUMMARY OF THE INVENTION

The invention provides processes for producing fatty acid alcohol esters using microbially produced alcohol and fatty acid, where esterification occurs in situ in fermentation medium.
Accordingly the invention provides a process for the production of fatty acid alcohol esters comprising:
   a) providing a microorganism which produces at least one alcohol;
   b) providing a microorganism which produces at least one free fatty acid or a microbial oil comprising at least one triacylglycerol, diacylglycerol, or monoacylglycerol, or mixtures thereof;
   c) growing the microorganism of a) in a medium comprising:
      i) optionally at least one fermentable carbon source;
      ii) a catalyst capable of esterifying free fatty acids with alcohol into fatty acid alkyl esters and optionally capable of hydrolyzing acylglycerols into free fatty acids; and
      iii) at least one free fatty acid derived from the microorganism of b);
   wherein fatty acid alcohol esters are formed extracellularly and in situ from esterification of the free fatty acids with the alcohol using the catalyst.
In another embodiment the invention provides a process for the production of fatty acid alcohol esters comprising:
   a) providing a microorganism which produces at least one alcohol;
   b) providing a microorganism which produces at least one fatty acid or a microbial oil comprising at least one triacylglycerol, diacylglycerol, or monoacylglycerol, or mixtures thereof;
   c) growing the microorganism of b) in a medium comprising:
      i) optionally at least one fermentable sugar;
      ii) a catalyst capable of esterifying free fatty acids with alcohol into fatty acid alkyl esters and optionally capable of hydrolyzing acylglycerols into free fatty acids; and
      iii) at least one alcohol derived from the microorganism of a);

wherein fatty acid alcohol esters are formed extracellularly and in situ from esterification of the free fatty acids with the alcohol using the catalyst.

In one aspect microorganisms of the invention include but are not limited to a yeast, bacteria, cyanobacteria, protists, microalgae, and filamentous fungi which may be anaerobic, facultative anaerobes, and an aerobic.

In another embodiment the invention the invention provides an alcohol fermentation process composition comprising:
a) a processed biomass comprising water and fermentable carbon source;
b) a catalyst capable of esterifying free fatty acids with alcohol into fatty acid alkyl esters and optionally capable of hydrolyzing acylglycerols into free fatty acids;
c) at least one alcohol produced by a microorganism;
d) free fatty acids produced by a microorganism;
e) optionally a microbial oil comprising at least one triacylglycerol, diacylglycerol, or monoacylglycerol, or mixtures thereof; and
f) fatty acid alcohol esters formed in situ from esterification of the free fatty acids with the alcohol using the catalyst.

### BRIEF DESCRIPTION OF THE SEQUENCE LISTING

The invention can be more fully understood from the following detailed description and the accompanying sequence descriptions, which form a part of this application.

The following sequences conform with 37 C.F.R. 1.821-1.825 (“Requirements for patent applications Containing Nucleotide Sequences and/or Amino Acid Sequence Disclosures—the Sequence Rules”) and consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO: 1 is the nucleotide sequence of the vector pYZ090 comprising chimeric genes containing 1) the yeast CUP1 promoter, Bacillus subtilis alsS coding region and CYC1 terminator, and 2) the yeast ILV5 promoter, Lactococcus lactis ilvC gene coding region, and ILV5 terminator. and TDH3 terminator.

SEQ ID NO: 2 is the nucleotide sequence of the vector pLH468 comprising chimeric genes containing 1) the S. cerevisiae FBA1 promoter, Streptococcus mutan silvD gene coding region, and FBA1 terminator, 2) the S. cerevisiae GPM1 promoter, codon optimized horse liver alcohol dehydrogenase coding region, and ADH1 terminator, and 3) the TDH3 promoter, Lactococcus lacti kivD gene codon-optimized coding region, and TDH3 terminator.

SEQ ID NO: 3 is the amino acid sequence of the Bacillus subtillis acetolactate synthase enzyme (alsS) protein.

SEQ ID NO: 4 is the amino acid sequence of the ilvC encoded Lactococcus lactis ketol-acid reductoisomerase enzyme.

SEQ ID NO: 5 is the amino acid sequence of the Streptococcus mutans ilvD encoded

dihydroxyacid dehydratase enzyme.

SEQ ID NO: 6 is the amino acid sequence of the Lactococcus lactis kivD encoded

branched-chain α-keto acid decarboxylase enzyme.

SEQ ID NO: 7 is the amino acid sequence of horse liver alcohol dehydrogenase enzyme.

SEQ ID NO: 8 is the amino acid sequence of the Achromobacter xylosoxidans sadB encoded alcohol dehydrogenase.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

In order to further define this invention, the following terms and definitions are herein provided.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, alternatively within 5% of the reported numerical value.

“Biomass” as used herein refers to a natural product containing hydrolysable polysaccharides that provide fermentable sugars, including any cellulosic or lignocellulosic material and materials comprising cellulose, and optionally further comprising hemicellulose, lignin, starch, oligosaccharides, disaccharides and/or monosaccharides. Biomass may also comprise additional components, such as protein and/or lipids. Biomass may be derived from a single source, or biomass can comprise a mixture derived from more than one source; for example, biomass may comprise a mixture of corn cobs and corn stover, or a mixture of grass and leaves. Biomass includes, but is not limited to, grains, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, rye, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof. Mash may be formed from biomass grains by any processing known in the art for processing the biomass for purposes of fermentation, such as by milling, treating and/or liquefying and comprises fermentable sugar and may comprise an amount of water. Fermentable sugar-containing hydrolysate may be formed from cellulosic or lignocellulosic biomass by any processing known in the art for processing the biomass for purposes of fermentation, such as by pretreating with acid, base, and/or mechanical size reduction, followed by enzymatic saccharification.

“Product alcohol” as used herein refers to any alcohol that can be produced by a microorganism in a fermentation process. Product alcohols include, but are not limited to, C₁ to C8 alkyl alcohols. It will be appreciated that C₁ to C8 alkyl alcohols include, but are not limited to, methanol, ethanol, propanol, butanol, and pentanol. “Alcohol” is also used herein with reference to a product alcohol.

“Butanol” as used herein refers with specificity to the butanol isomers 1-butanol (1-BuOH), 2-butanol (2-BuOH) and/or isobutanol (iBuOH or i-BuOH or I-BUOH, also known as 2-methyl-1-propanol), either individually or as mixtures thereof.

“Propanol” as used herein refers to the propanol isomers isopropanol (2-propanol) or 1-propanol.

“Pentanol” as used herein refers to the pentanol isomers 1-pentanol, 3-methyl-1-butanol, 2-methyl-1-butanol, 2,2-dimethyl-1-propanol, 3-pentanol, 2-pentanol, 3-methyl-2-butanol, or 2-methyl-2-butanol.

“Product fatty acid” refers herein to fatty acids and derivatives of fatty acids synthesized by a microbial system. Product fatty acids may be in the form of free fatty acids or derivatives thereof, such as glycerolipids (composed mainly of mono-, di- and tri-acylglycerols), saccharolipids, and phospholipids (for example, phosphatadylcholine). Product fatty acids are typically found in the cell in membranes, micelles and/or oil droplets.

“Acylglycerols” refers to mono-, di-, and triacylglycerols.

“Fermentable carbon source” as used herein means a carbon source capable of being metabolized by the microorganisms disclosed herein for the production of alcohol and/or product fatty acids. Suitable fermentable carbon sources include, but are not limited to, monosaccharides, such as glucose, xylose and fructose; disaccharides, such as lactose or sucrose; oligosaccharides; glycerol, and mixtures thereof.

“Feedstock” as used herein means a feed in a fermentation process, the feed containing a fermentable carbon source with or without undissolved solids, and where applicable, the feed containing the fermentable carbon source before or after the fermentable carbon source has been liberated from starch or obtained from the break down of complex polysaccharides by further processing, such as by liquefaction, saccharification, or other process. Feedstock includes or is derived from biomass.

“Sugars” as used herein refers to oligosaccharides, disaccharides and/or monosaccharides.

“Fermentable sugars” as used herein refers to sugars capable of being metabolized by the microorganisms disclosed herein for the production of fermentative products, including alcohols and fatty acids.

Undissolved solids” as used herein means non-fermentable portions of feedstock, for example germ, fiber, and gluten.

The term “medium” refers to an aqueous milieu used to support the growth of microorganisms. A “medium” may be a “fermentation medium” or “fermentation broth” which will refer to a medium containing a mixture of water, sugars, dissolved solids, microorganisms producing alcohol and/or fatty acid products and other constituents of the material held in a fermentation vessel in which product is being made by the biocatalyst.

“In situ” as used herein refers to taking place in fermentation broth.

“Fermentation vessel” as used herein means the vessel in which the fermentation reaction by which product, such as alcohol or fatty acid, is made from sugars is carried out.

“Saccharification vessel” as used herein means the vessel in which saccharification (i.e., the break down of oligosaccharides into monosaccharides) is carried out. Where fermentation and saccharification occur simultaneously, the saccharification vessel and the fermentation vessel may be the same vessel.

“Liquefaction vessel” as used herein means the vessel in which liquefaction is carried out. Liquefaction is the process in which oligosaccharides are liberated from a feedstock. In embodiments where the feedstock is corn grain, oligosaccharides are liberated from the corn starch content during liquefaction.

The term “separation” as used herein is synonymous with “recovery” and refers to removing a chemical compound from an initial mixture to obtain the compound in greater purity or at a higher concentration than the purity or concentration of the compound in the initial mixture.

“Microbial oils” are those oils produced by microorganisms such as algae, oleaginous yeasts and filamentous fungi. The term “oil” refers to a nonpolar substance that is liquid at 25° C. and hydrophobic but soluble in organic solvents. Microbial oils contain fatty acids that are in the form of free fatty acids, or as glycerolipids (composed mainly of mono-, di- and tri-acylglycerols), saccharolipids, and phospholipids (for example, phosphatadylcholine).

The term “fatty acid” as used herein refers to a carboxylic acid having an aliphatic chain of C₆ to C₂₂ carbon atoms, which is either saturated or unsaturated. When referred to herein as “free” the fatty acids are unesterified. The structure of a fatty acid is represented by a simple notation system of “X:Y”, where X is the total number of carbon [“C”] atoms in the particular fatty acid and Y is the number of double bonds.

The term “oleaginous” refers to those organisms that tend to store their energy source in the form of oil (Weete, In: Fungal Lipid Biochemistry, 2nd Ed., Plenum, 1980). It is not uncommon for oleaginous microorganisms to accumulate in excess of about 20% of their dry cell weight as oil. Examples of oleaginous yeast include, but are no means limited to, the following genera: Yarrowia, Candida, Rhodotorula, Rhodosporidium, Cryptococcus, Trichosporon and Lipomyces.

The term “butanol biosynthetic pathway” as used herein refers to an enzyme pathway to produce 1-butanol, 2-butanol, or isobutanol.

The term “1-butanol biosynthetic pathway” as used herein refers to an enzyme pathway to produce 1-butanol from acetyl-coenzyme A (acetyl-CoA).

The term “2-butanol biosynthetic pathway” as used herein refers to an enzyme pathway to produce 2-butanol from pyruvate.

The term “isobutanol biosynthetic pathway” as used herein refers to an enzyme pathway to produce isobutanol from pyruvate.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign gene” or “heterologous gene” refers to a gene not normally found in the host organism, but that is introduced into the host organism by gene transfer. Foreign genes can comprise native genes inserted into a non-native organism, or chimeric genes.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Codon optimization is within the ordinary skill in the art.

The term “polynucleotide” is intended to encompass a singular nucleic acid as well as plural nucleic acids, and refers to a nucleic acid molecule or construct, e.g., messenger RNA (mRNA) or plasmid DNA (pDNA). As used herein, a “gene” is a polynucleotide. A polynucleotide can contain the nucleotide sequence of the full-length cDNA sequence, or a fragment thereof, including the untranslated 5′ and 3′ sequences and the coding sequences. The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. “Polynucleotide” embraces chemically, enzymatically, or metabolically modified forms.

A polynucleotide sequence may be referred to as “isolated,” in which it has been removed from its native environment. For example, a heterologous polynucleotide encoding a polypeptide or polypeptide fragment having dihydroxy-acid dehydratase activity contained in a vector is considered isolated for the purposes of the present invention. Further examples of an isolated polynucleotide include recombinant polynucleotides maintained in heterologous host cells or purified (partially or substantially) polynucleotides in solution. Isolated polynucleotides or nucleic acids according to the present invention further include such molecules produced synthetically. An isolated polynucleotide fragment in the form of a polymer of DNA may be comprised of one or more segments of cDNA, genomic DNA or synthetic DNA.

The term “polypeptide” refers to any chain or chains of two or more amino acids, and does not refer to a specific length of the product. As used herein, the term “polypeptide” is intended to encompass a singular “polypeptide” as well as plural “polypeptides,” and refers to a molecule composed of monomers (amino acids) linearly linked by amide bonds (also known as peptide bonds). Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. A polypeptide may be derived from a natural biological source or produced by recombinant technology, but is not necessarily translated from a designated nucleic acid sequence. It may be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

The present invention satisfies the need for alternative processes for producing biodiesel fuel in a sustainable manner, through the use of alcohols and fatty acids produced by microorganisms using renewable resources. The present in situ esterification process occurs in fermentation medium of at least one microbial biocatalyst producing alcohol and/or fatty acid. The resulting fatty acid alkyl esters, also called fatty acid alcohol esters, may be used in commercial products including as biodiesel fuel.

Decreasing the amount of water present in a reaction system, or employing a reaction system that substitutes an organic solvent for water, has typically been necessary for esterification of fatty acids by alcohols when using enzymes catalysts such as lipases. Described herein is the finding that lipase enzymes can efficiently catalyze the esterification of a product alcohol with a fatty acid in a fermentation broth, which thereby allows in situ esterification.

In the present processes, fatty acids and alcohols are produced by microorganisms using renewable resources In one embodiment microorganisms producing fatty acids, which may be produced as oil, and/or alcohol are photosynthetic, for example, algae. In one embodiment, one or more fermentable sugar (used as a carbon source in fermentation medium used to support microorganism production is provided from one or more renewable feedstocks. Feedstocks that may be used include grain such as rye, wheat, or corn that may be one or more components of a fractionated biomass or milled, unfractionated biomass such as corn. The starch of grain feedstock is liquefied by hydrolysis to produce sugars by any method known in the art such as by acid, acid-enzyme, and/or enzyme processes. A typically used enzyme is alpha-amylase. For example, a corn mash slurry containing sugars, undissolved solids and oil that is produced by liquefying corn grain can be used as a source of fermentable sugars in fermentation medium.

In addition feedstocks that may be used to produce fermentable sugars include cellulosic and/or lignocellulosic biomass, described above, including typically used types of biomass such as corn cob, corn stover, grasses such as switch grass, and sugar cane bagasse. Cellulosic and/or lignocellulosic biomass may be processed to obtain a hydrolysate containing fermentable sugars by any method known to one skilled in the art. Typically the biomass is pretreated using physical and/or chemical treatments, and saccharified enzymatically. Physical and chemical treatments include, but are not limited to, grinding, milling, cutting, base treatment such as with ammonia or NaOH, and acid treatment. Particularly useful is a low ammonia pretreatment where biomass is contacted with an aqueous solution comprising ammonia to form a biomass-aqueous ammonia mixture where the ammonia concentration is sufficient to maintain alkaline pH of the biomass-aqueous ammonia mixture but is less than about 12 weight percent relative to dry weight of biomass, and where dry weight of biomass is at least about 15 weight percent solids relative to the weight of the biomass-aqueous ammonia mixture, as disclosed in co-pending and commonly owned US Patent Application Publication US20070031918A1, which is herein incorporated by reference.

Enzymatic saccharification typically makes use of an enzyme consortium for breaking down cellulose and hemicellulose to produce a hydrolysate containing sugars including glucose, xylose, and arabinose. Saccharification enzymes are reviewed in Lynd, L. R., et al. (Microbiol. Mol. Biol. Rev., 66:506-577, 2002). For saccharification, typically an enzyme consortium is used that includes one or more glycosidases found in the enzyme classification EC 3.2.1.x. Commercially available saccharification enzymes include Spezyme® CP cellulase, Multifect® xylanase, Accelerase® 1500, and Accellerase® DUET (Danisco U.S. Inc., Genencor International, Rochester, N.Y.). In addition, saccharification enzymes may be unpurified and provided as a type of cell extract or whole cell preparation. The enzymes may be produced using recombinant microorganisms that have been engineered to express multiple saccharifying enzymes.

Fermentation medium containing sugars from mash or hydrolysate is used to support growth of a microorganism, or biocatalyst, that produces alcohol and/or fatty acids. The fermentation medium may contain additional components such as additional sugars, other nutrients, salts, and factors required for growth and production by the specific biocatalyst to be used for product production, as well known to one skilled in the art. Glycerol may be used as a carbon source in fermentation medium in addition to sugars, or as an alternative to sugars. Supplements may include, for example, yeast extract, specific amino acids, phosphate, nitrogen sources, salts, and trace elements. Components required for production of a specific product made by a specific biocatalyst may also be included, such as an antibiotic to maintain a plasmid or a cofactor required in an enzyme catalyzed reaction. When a biocatalyst is used that has a butanol biosynthetic pathway and/or reduced or eliminated expression of pyruvate decarboxylase, supplementation of a 2-carbon substrate, e.g., ethanol, may be needed for survival and growth of the biocatalyst.

It will be appreciated that in combining the steps of fermentation and saccharification that saccharification may occur concurrently with fermentation in a simultaneous saccharification and fermentation (SSF) process. In this method, saccharification enzymes are included in the fermentation medium for hydrolysis, or further hydrolysis, of a treated biomass feedstock.

Microorganisms used to produce the target products alcohol and/or fatty acids may include bacteria, protists, cyanobacteria, filamentous fungi, yeasts, and algae, particularly microalgae. Microalgae may include diatoms and green algae. The microorganism may produce the target product naturally using an endogenous metabolic pathway, or it may be genetically engineered for production, or for increased production, of the target product using a recombinant metabolic pathway.

A microorganism producing alcohol and/or fatty acid target products may be an anaerobe, a facultative anaerobe, or an aerobe. Fermentation may be aerobic or anaerobic.

Alcohols produced by the present microorganisms include, but are not limited to, methanol, ethanol, propanol (iso-propanol and 1-propanol), butanol (1-butanol, 2-butanol, and isobutanol). Biocatalysts producing alcohols may be wild type microorganisms or recombinant microorganisms. Fermentation of carbohydrates to acetone, butanol, and ethanol (ABE fermentation) by solventogenic Clostridia is well known (Jones and Woods (1986) Microbiol. Rev. 50:484-524). Yeasts naturally produce ethanol, and may be genetically engineered for improved ethanol production (Alper et al. (2010) Science 314:1565-1568). Fermentative oxidation of methane by methanotrophic bacteria, for example Methylosinus trichosporium, produces methanol.

Microorganisms useful for the production of alcohol may be either wildtyp or recombinant and for example, may include, but not be limited to, a member of the genera Clostridium, Zymomonas, Escherichia, Salmonella, Serratia, Erwinia, Klebsiella, Shigella, Rhodococcus, Pseudomonas, Bacillus, Lactobacillus, Streptomyces, Enterococcus, Alcaligenes, Klebsiella, Paenibacillus, Arthrobacter, Corynebacterium, Brevibacterium, Synechococcus, Thermoanaerobacterium, Schizosaccharomyces, Kluyveromyces, Yarrowia, Pichia, Candida, Hansenula, Issatchenkia, or Saccharomyces. In one embodiment, recombinant microorganisms can be selected from the group consisting of Escherichia coli, Lactobacillus plantarum, Zymomonas mobilis, and Saccharomyces cerevisiae. In one embodiment, the recombinant microorganism is a yeast. In one embodiment, the recombinant microorganism is a crabtree-positive yeast selected from Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Zygosaccharomyces rouxii, and Candida glabrata.

For example, the production of butanol utilizing fermentation by a microorganism, as well as microorganisms which produce butanol, is disclosed, for example, in U.S. Patent Publication. No. 2009/0305370, herein incorporated by reference. In some embodiments, microorganisms comprise a butanol biosynthetic pathway. In different embodiments, at least one, at least two, at least three, or at least four polypeptides catalyzing substrate to product conversions of a pathway are encoded by heterologous polynucleotides in the microorganism. In some embodiments, all polypeptides catalyzing substrate to product conversions of a pathway are encoded by heterologous polynucleotides in the microorganism. In some embodiments, the microorganism comprises a reduction or elimination of pyruvate decarboxylase activity. Microorganisms substantially free of pyruvate decarboxylase activity are described in US Application Publication No. 20090305363, herein incorporated by reference. Microorganisms substantially free of an enzyme having NAD-dependent glycerol-3-phosphate dehydrogenase activity such as GPD2 are also described therein.

Suitable biosynthetic pathways for production of butanol are known in the art, and certain suitable pathways are described herein. Certain suitable proteins having the ability to catalyze indicated substrate to product conversions of said butanol biosynthetic pathways are described herein and other suitable proteins are provided in the art. For example, US Published Patent Application Nos. US 2008/0261230, US 2009/0163376 and US 2010/0197519, incorporated herein by reference, describe acetohydroxy acid isomeroreductases; US Patent Application No. US20100081154, incorporated by reference, describes dihydroxyacid dehydratases; an alcohol dehydrogenase is described in US Published Patent Application US 2009/0269823, incorporated herein by reference.

It will be appreciated by one skilled in the art that many levels of sequence identity are useful in identifying polypeptides, from other species, wherein such polypeptides have the same or similar function or activity and are suitable for use in the recombinant microorganisms described herein. Useful examples of percent identities include, but are not limited to: 75%, 80%, 85%, 90%, or 95%, or any integer percentage from 75% to 100% may be useful in describing the present invention, such as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

### 1-Butanol Biosynthetic Pathway

A biosynthetic pathway for the production of 1-butanol as well as suitable polypeptides and polynucleotides encoding such polypeptides that may be used is described by Donaldson et al. in U.S. Patent Application Publication No. US 2008/0182308, incorporated herein by reference. This biosynthetic pathway comprises the following substrate to product conversions:

a) acetyl-CoA to acetoacetyl-CoA, which may be catalyzed, for example, by acetyl-CoA acetyltransferase;

b) acetoacetyl-CoA to 3-hydroxybutyryl-CoA, which may be catalyzed, for example, by 3-hydroxybutyryl-CoA dehydrogenase;

c) 3-hydroxybutyryl-CoA to crotonyl-CoA, which may be catalyzed, for example, by crotonase;

d) crotonyl-CoA to butyryl-CoA, which may be catalyzed, for example, by butyryl-CoA dehydrogenase;

e) butyryl-CoA to butyraldehyde, which may be catalyzed, for example, by butyraldehyde dehydrogenase; and

f) butyraldehyde to 1-butanol, which may be catalyzed, for example, by 1-butanol dehydrogenase

In some embodiments, the 1-butanol biosynthetic pathway comprises at least one gene, at least two genes, at least three genes, at least four genes, or at least five genes that is/are heterologous to the yeast cell. In some embodiments, the recombinant host cell comprises a heterologous gene for each substrate to product conversion of a 1-butanol biosynthetic pathway.

### 2-Butanol Biosynthetic Pathway

Biosynthetic pathways for the production of 2-butanol as well as suitable polypeptides and polynucleotides encoding such polypeptides that may be used are described by Donaldson et al. in U.S. Patent Application Publication Nos. US 2007/0259410 and US 2007/0292927, and in PCT Publication WO 2007/130521, all of which are incorporated herein by reference. One 2-butanol biosynthetic pathway comprises the following substrate to product conversions:

a) pyruvate to alpha-acetolactate, which may be catalyzed, for example, by acetolactate synthase;

b) alpha-acetolactate to acetoin, which may be catalyzed, for example, by acetolactate decarboxylase;

c) acetoin to 2,3-butanediol, which may be catalyzed, for example, by butanediol dehydrogenase;

d) 2,3-butanediol to 2-butanone, which may be catalyzed, for example, by butanediol dehydratase; and

e) 2-butanone to 2-butanol, which may be catalyzed, for example, by 2-butanol dehydrogenase.

In some embodiments, the 2-butanol biosynthetic pathway comprises at least one gene, at least two genes, at least three genes, or at least four genes that is/are heterologous to the yeast cell. In some embodiments, the recombinant host cell comprises a heterologous gene for each substrate to product conversion of a 2-butanol biosynthetic pathway.

### Isobutanol Biosynthetic Pathway

Biosynthetic pathways for the production of isobutanol as well as suitable polypeptides and polynucleotides encoding such polypeptides that may be used are described in U.S. Patent Application Publication No. US 2007/0092957 and PCT Publication WO 2007/050671, incorporated herein by reference. One isobutanol biosynthetic pathway comprises the following substrate to product conversions:

a) pyruvate to acetolactate, which may be catalyzed, for example, by acetolactate synthase;

b) acetolactate to 2,3-dihydroxyisovalerate, which may be catalyzed, for example, by acetohydroxy acid reductoisomerase;

c) 2,3-dihydroxyisovalerate to α-ketoisovalerate, which may be catalyzed, for example, by acetohydroxy acid dehydratase;

d) α-ketoisovalerate to isobutyraldehyde, which may be catalyzed, for example, by a branched-chain keto acid decarboxylase; and

e) isobutyraldehyde to isobutanol, which may be catalyzed, for example, by a branched-chain alcohol dehydrogenase.

Suitable polypeptide sequences that encode enzymes which catalyze the substrate to product conversions of the isobutanol biosynthetic pathway as well as E.G. numbers corresponding to suitable enzymes for the indicated pathway steps, include, but are not limited to those in Tables AA and BB. Suitable enzymes associated with the given E.G. numbers will be readily available to those of skill in the art, for example through the BRENDA database (http://www.brenda-enzymes.org/).

TABLE 1
Example polypeptides

| | | SEQ |
|---|---|---|
| | | ID |
| Pathway step | Enzyme | NO: |
| a) pyruvate to acetolactate | Bacillus subtilis alsS (acetolactate | 3 |
| | synthase) | |
| b) acetolactate to 2,3- | Lactococcus lactis ilvC (ketol-acid | 4 |
| dihydroxyisovalerate | reductoisomerase, “KARI”) | |
| c) 2,3-dihydroxyisovalerate to α- | Streptococcus mutans ilvD (dihydroxyacid | 5 |
| ketoisovalerate | dehydratase, “DHAD”) | |
| d) α-ketoisovalerate to | Lactococcus lactis kivD (branched-chain | 6 |
| isobutyraldehyde | α-keto acid decarboxylase), codon | |
| | optimized | |
| e) isobutyraldehyde to isobutanol | horse liver alcohol dehydrogenase (“ADH”) | 7 |
| f) isobutyraldehyde to isobutanol | Achromobacter xylosoxidans sadB | 8 |

TABLE 2
E.C. numbers of enzymes used to perform pathway steps

| Pathway step | E.C. number |
|---|---|
| a) pyruvate to acetolactate | 2.2.1.6 |
| b) acetolactate to 2,3-dihydroxyisovalerate | 1.1.1.86 |
| c) 2,3-dihydroxyisovalerate to | 4.2.1.9 |
| α-ketoisovalerate | |
| d) α-ketoisovalerate to isobutyraldehyde | 4.1.1.72 or 4.1.1.1 |
| e) isobutyraldehyde to isobutanol | 1.1.1.265, 1.1.1.1 or 1.1.1.2 |

Provided herein are recombinant microorganisms comprising an isobutanol biosynthetic pathway comprising steps a)-e) (above) wherein at least one of the enzymes selected from the group of the enzyme catalyzing step c) and the enzyme catalyzing step e) is encoded by a heterologous polynucleotide integrated into the chromosome of the microorganism. In embodiments, both an enzyme catalyzing step c) is encoded by a heterologous polynucleotide integrated into the chromosome of the microorganism, and enzyme catalyzing step c) is encoded by a heterologous polynucleotide integrated into the chromosome of the microorganism.

Provided herein are polynucleotides suitable for recombinant microorganisms comprising a butanol biosynthetic pathway such as an isobutanol biosynthetic pathway. Such polynucleotides include the coding region of the alsS gene from Bacillus subtilis (nt position 457-2172 of SEQ ID NO:1) and the ilvC gene from Lactococcus lactis (nt 3634-4656 of SEQ ID NO:1) as well as plasmids comprising either or both. Also suitable is a chimeric gene having the coding region of the alsS gene from Bacillus subtilis (nt position 457-2172 of SEQ ID NO:1) expressed from the yeast CUP1 promoter (nt 2-449 of SEQ ID NO:1) and followed by the CYC1 terminator (nt 2181-2430 of SEQ ID NO:1) for expression of ALS, and a chimeric gene having the coding region of the ilvC gene from Lactococcus lactis (nt 3634-4656 of SEQ ID NO:1) expressed from the yeast ILV5 promoter (2433-3626 of SEQ ID NO:1) and followed by the ILV5 terminator (nt 4682-5304 of SEQ ID NO:1) for expression of KARI, as well as plasmids comprising either or both chimeric genes.

Suitable polynucleotides include the coding region of the ilvD gene from Streptococcus mutans (nt position 3313-4849 of SEQ ID NO:2), the coding region of codon optimized horse liver alcohol dehydrogenase (nt 6286-7413 of SEQ ID NO:2), the coding region of the codon-optimized kivD gene from Lactococcus lactis (nt 9249-10895 of SEQ ID NO:2) as well as plasmids comprising any or all or any combination thereof. Also suitable is a chimeric gene having the coding region of the ilvD gene from Streptococcus mutans (nt position 3313-4849 of SEQ ID NO:2) expressed from the S. cerevisiae FBA1 promoter (nt 2109-3105 of SEQ ID NO:2) followed by the FBA1 terminator (nt 4858-5857 of SEQ ID NO:2) for expression of DHAD; a chimeric gene having the coding region of codon optimized horse liver alcohol dehydrogenase (nt 6286-7413 of SEQ ID NO:2) expressed from the S. cerevisiae GPM1 promoter (nt 7425-8181 of SEQ ID NO:2) followed by the ADH1 terminator (nt 5962-6277 of SEQ ID NO:2) for expression of ADH; and a chimeric gene having the coding region of the codon-optimized kivD gene from Lactococcus lactis (nt 9249-10895 of SEQ ID NO:2) expressed from the TDH3 promoter (nt 10896-11918 of SEQ ID NO:2) followed by the TDH3 terminator (nt 8237-9235 of SEQ ID NO:2) for expression of KivD as well as plasmids containing any, all, or any combination of such chimeric genes. In addition, suitable polynucleotides include those having at least about 75% identity to the coding regions and chimeric genes specified, as well as plasmids comprising such polynucleotides.

In some embodiments, the isobutanol biosynthetic pathway comprises at least one gene, at least two genes, at least three genes, or at least four genes that is/are heterologous to the yeast cell. In some embodiments, the recombinant host cell comprises a heterologous gene for each substrate to product conversion of an isobutanol biosynthetic pathway.

Genetically modified strains of E. coli may be used as biocatalysts for ethanol production. The ethanol biosynthesis pathway of Zymomonas has been engineered in E. coli through expression of Z. mobilis pyruvate decarboxylase (pdc) and alcohol dehydrogenase II (adhB; Ohta et al. (1991) Appl. Environ. Microbiol. 57:893-900). Expression of an NADH-insensitive citrate synthase (citZ) from Bacillus subtilis improved ethanol production (Underwood et al. (2002) Appl. Environ. Microbiol. 68:1071-1081).

E. coli strains that are genetically modified for production of 1-butanol and 1-propanol are described in Shen and Liao ((2008) Metab. Eng. 10:3120320). 1-propanol was made from 2-ketobutyrate through expression of 2-ketoacid decarboxylase (Kivd) from Lactococcus lactis and alcohol dehydrogenase 2 (ADH2) from Saccharomyces cerevisiae. E. coli has been engineered for production of isopropanol as described in Hahnai et al. ((2007) Appl. Environ. Microbiol. 73:7814-7818). An effective engineered biosynthetic pathway included acetyl-coenzyme A acetyltransferase from Clostridium acetobutylicum (thl), acetoacetyl-CoA transferase from E. coli (atoAD), acetoacetate decarboxylase from C. acetobutylicum (adc), and secondary alcohol dehydrogenase from C. beijerinckii (adh).

Any of these alcohol producing strains, or additional microorganisms engineered to express these or other alcohol biosynthetic pathways may also be used in the present process. Engineering of microorganisms is well known to one skilled in the art. For example, one skilled in the art is familiar with the use of chimeric genes for protein expression, replicating or integrating vectors for transformation, homologous recombination for targeted gene introduction or inactivation, including the use of selection markers, site-specific recombination, and many other tools for genetic engineering.

Zymomonas mobilis naturally produces ethanol, and improved ethanol production by Z. mobilis has been accomplished by genetic engineering of this microorganism. Z. mobilis has been engineered to utilize xylose for ethanol production (U.S. Pat. No. 5,514,583, U.S. Pat. No. 5,712,133, WO 95/28476, Feldmann et al. (1992) Appl. Microbiol. Biotechnol. 38: 354-361, Zhang et al. (1995) Science 267:240-243), a sugar found in biomass hydrolysate. Ethanol has been produced by genetically modified Zymomonas in lignocellulosic biomass hydrolysate fermentation media (US 2007/0031918). Genetically modified strains of Z. mobilis with improved xylose utilization and/or production of ethanol are disclosed in U.S. Pat. No. 7,223,575, U.S. Pat. No. 7,741,119, US 2009/0203099, US 2009/0246846, and WO2010/075241, which are herein incorporated by reference. Any of the disclosed strains, including for example ATCC31821/pZB5, ZW658 (ATCC #PTA-7858), ZW800, ZW801-4, ZW801-4:: ΔhimA, AcR#3, ZW705, or other ethanol-producing strains of Zymomonas may be used to produce ethanol in the present process.

Microorganisms that may be used to produce fatty acids include bacteria, microalgae, protists, filamentous fungi and yeast. In one embodiment the microorganism is oleaginous, such as oleaginous yeast and microalgae including diatoms and green algae. Oleaginous microorganisms naturally produce oil, and typically contain at least about 20% of dry cell weight as oil. Microorganisms that may be used produce fatty acids naturally, or they may be engineered to produce higher amounts of fatty acids and/or specific fatty acids, in each case using a metabolic pathway in the microorganism. In one embodiment microorganisms used to produce fatty acids, which may be in oil, grow photosynthetically using CO₂ and light. In another embodiment microorganisms used to produce fatty acids, which may be in oil, grow in fermentation medium containing at least one carbon source such as sugar and/or glycerol. For example, fatty acids may be produced in substantial amounts naturally by microorganisms such Euglena gracilis (a unicellular protist), Rhodococcus opacus (a bacteria), Botryococcus braunii (a microalga), Mortierella isabellina (an oleaginous Zygomycete fungus), Yarrowia lipolytica (an oleaginous yeast), Lipomyces starkeyi (a fungus), Chlorella protothecoides (a microalga), Rhodosporidium toruloides (an oleaginous yeast), Trochosporon fermentans (an oleaginous yeast), and the algae-like protists Thraustochytrids. Algae may be used such as of the genus Chlamydomonas, Dunaliella, Haematococcus, Scenedesmus, Chlorella, Cyclotella, Hantzschia, Nitzschia, Ankistrodesmus. The bacteria may be cyanobacteria or other bacteria of the genus Synechocystis, Synechococcus or Arhtospira. Oleaginous yeast include Yarrowia, Candida, Rhodotorula, Rhodosporidium, Cryptococcus, Saccharomyces, Hanselula, Pichia, Aspergillis Mortierella, Conidiobolus, Pythium, Phytophathora, Penicillium, Porphyridium, Coidosporium, Mucor, Fusarium, Entomophthora., Trichosporon and Lipomyces. Oleaginous microalgae include, for example, species of Scenedesmus, Chlorella, Cyclotella, Hantzschia, Nitzschia, and Ankistrodesmus.

Microorganisms used for fatty acid production may be engineered to have improved production such as having improved performance under typical production conditions. Microorganisms may be genetically engineered to enhance endogenous fatty acid biosynthetic pathways, and/or to introduce non-endogenous fatty acid biosynthetic pathways. Methods for genetic engineering of microorganisms are well known to one skilled in the art as noted above.

There are multiple pathways for fatty acid biosynthesis in microorganisms, as described for example in Tehlivets et al. ((2007) Biochim. Biophys. Acta 1771:255-270), and Inui et al. ((1984) Eur. J. Biochem. 142:121-126)), including malonyl-CoA dependent and malonyl-CoA-independent pathways. Although the molecular structures of the enzymes involved in malonyl-CoA dependent fatty acid synthesis and fatty acid elongation are quite diverse between different prokaryotic and eukaryotic species, the reaction mechanisms are essentially the same in all types of cells. In an initial step, acetyl-CoA is carboxylated by the addition of CO₂ to malonyl-CoA, by the enzyme acetyl-CoA carboxylase (ACC; encoded by ACC1 and HFA1 in yeast). Biotin is an essential cofactor in this reaction, and is covalently attached to the ACC apoprotein, by the enzyme biotin:apoprotein ligase (encoded by BPL1/ACC2 in yeast). ACC (EC 6.4.1.2) is a trifunctional enzyme, harboring a biotin carboxyl carrier protein (BCCP) domain, a biotin-carboxylase (BC) domain, and a carboxyl-transferase (CT) domain. In most bacteria, these domains are expressed as individual polypeptides and assembled into a heteromeric complex. In contrast, eukaroytic ACC, including mitochondrial ACC variants (Hfa1 in yeast) harbor these functions on a single polypeptide. Malonyl-CoA produced by ACC serves as a two carbon donor in a cyclic series of reactions catalyzed by fatty acid synthase, FAS, and elongases.

In malonyl-CoA independent pathways, fatty acids are directly polymerized from acyl-CoA units that serve as both primers and carbon chain donors without prior activation through an energy-requiring carboxylation step that typically is catalyzed by the acetyl-CoA carboxylase to transform acetyl-CoA into malonyl-CoA to serve as a C2-donor. A malonyl-CoA independent pathway may be used to achieve fatty acid production under anaerobic conditions.

In most bacteria, and also in mitochondria or in chloroplasts of eukaryotic cells, the reactions associated with saturated fatty acid synthesis are catalyzed by dissociated, individual gene products (type II FAS systems), similarly to the initial ACC reaction. In contrast, the individual functions involved in cytosolic fatty acid synthesis of eukaryotic cells are represented as discrete domains on a single or on two different polypeptide chains, respectively. For example yeast cytosolic fatty acid synthase is composed of two subunits, Fas1 (β subunit) and Fas2 (α subunit) which are organized as a hexameric α6β6 complex. Fas1 harbors acetyl transferase, enoyl reductase, dehydratase, and malonyl-palmitoyl transferase activities; Fas2 contains acyl carrier protein, 3-ketoreductase, 3-ketosynthase and phoshopantheteine transferase activities. The corresponding enzymes in E. coli, for example, are encoded by fabI, fabD, fabG, and fabH/F/B. Mitochondrial fatty acid synthesis in yeast is carried out by a type II FAS system, harboring the individual enzymatic activities on distinct polypeptides: Acp1, acyl-carrier protein which carries the prosthetic phosphopantetheine group; Cem1, β-ketoacyl-ACP synthase; Oar1,3-oxoacyl-[acyl-carrier-protein] reductase; Htd-2,3-hydroxyacyl-thioester dehydratase; Etr1, enoyl-ACP reductase. Ppt2 functions as the phosphopantetheine: protein transferase, catalyzing the attachment of the phosphopantetheine prosthetic group to the apoACP.

Typically 3-oxoacyl-[acyl-carrier-protein] reductase and enoyl-ACP reductase in type I or type II FAS systems require NADPH as a cofactor in fatty acid biosynthesis. However, enzymes may be used that can also use NADH and do not depend on NADPH as the source of reduction equivalents for the production of fatty acids. This may be achieved by changing the cofactor-preferences of the enzymes by means of protein engineering. However, also naturally occurring enzymes and their homologues can be used. One class of such preferable NADH-utilizing enzymes that may be used is 3-oxoacyl-[acyl-carrier-protein] reductase enzyme, as for example described for plastids from the mesocarp of Avocado fruit by Caughey and KekwicK ((1982) Eur. J. Biochem. 123:553-561). Another class of such preferable enzymes that may be used are trans-2-enoyl-CoA reductases that catalyze the NAD(P)H-dependent synthesis of acyl-CoA from enoyl-CoA as described for mitochondrial wax ester biosynthesis in Euglena gracilis by Hoffmeister et al. ((2005) J. Biol. Chem. 280:4329-4338).

Short chain fatty acids may be synthesized utilizing NAD(P)H-dependent short chain 3-hydroxyacyl-CoA dehydrogenases and acyl-CoA dehydrogenases. Suitable 3-hydroxyacyl-CoA dehydrogenases can be found in C. acetobutylicum (Boynton et al. (1996) J. Bacteriol. 178:3015-3024) or by means of sequence homology searches. Suitable acyl-CoA dehydrogenases can be found in Clostridium kluyveri (La Roche et al. (1971) Physiol. Chem. 352:399-402), C. acetobutylicum (Boynton, ibid.) or M. elsdenii (Engel and Massey (1971) Biochem. J. 125:879-887) or by means of sequence homology searches. It is well understood by one skilled in the art that the described short-chain 3-hydroxyacyl-CoA dehydrogenases and acyl-CoA dehydrogenases can be modified by means of protein engineering to efficiently catalyze reactions with compounds containing more than four carbon atoms.

NADH-utilizing enzymes and their homologues are favorable especially under anaerobic conditions, to link NADH production in glycolysis and the acetyl-CoA generation from pyruvic acid to fatty acid biosynthesis in a redox-neutral balance. However, if either one or both of the biosynthetic steps in fatty acid formation, the 3-oxoacyl-[acyl-carrier-protein] reductase/3-hydroxyacyl-CoA dehydrogenase or the trans-2-enoyl-CoA reductase/acyl-CoA dehydrogenase reaction are NADP(H)-dependent or show respectively mixed cofactor dependencies, reactions in substrate catabolism that are coupled to the production of reduced redox metabolites like e.g. the conversion of glyceraldehyde-3-phosphate to either 1,3-bisphosphoglycerate or 3-phosphoglycerate or the conversion of pyruvate to acetyl-CoA, can be genetically engineered to be catalyzed by enzymes that produce either NADPH or NADH or both in a mixed ratio to match the cofactor requirements of the fatty acid biosynthesis pathway.

Fatty acid production can be increased by modifications of the biosynthesis pathway. Such modifications include up-regulating and down-regulating expression of enzymes required for biosynthesis (Lu et al. (2008) Metab. Eng. 10:333-339), as well as replacing or augmenting the activity of homologous enzymes by heterologous enzymes with preferred catalytic properties. Preferred catalytic properties refer to features of an enzyme that result in an increased in vivo specific activity, due to e.g. reduced inhibition by inhibitors, increased activity by activators, improved k_{cat} values or favorable affinity constants with respect to in vivo substrate and product concentrations encountered by the enzyme.

In addition to engineering increased production of fatty acids and/or lipids, also the type of the fatty acids with respect to suitability as diesel fuel feedstock can be optimized. The carbon chain length and degree of unsaturation of the fatty acids affect the cold flow and oxidative stability properties of a biodiesel fuel. Typically most fatty acids of microbes have a carbon chain length between 14 and 20; some major species are 16:1, 16:0, and 18:1. For diesel production, preferred fatty acids are lauric acid (12:0) and myristic acid (14:0). The chain lengths of fatty acids can be manipulated by using suitable acyl-ACP thioesterases. Acyl-ACP thioesterases release the fatty acid chain from the fatty acid synthase. There are several acyl-ACP thioesterases from a variety of organisms that are specific for certain fatty acid chain lengths. For example, a thioesterase from Umbellularia californica may be expressed to increase production of lauric acid (Voelker and Cavies (1994) J. Bacteriol. 176:7320-7327), a thioesterase from Cinnamomum camphorum may be expressed to increase production of myristic acid (Yuan et al. (1995) Proc. Nat. Acad. Sci. 92:10639-10643), and a thioesterase from Cuphea hookeriana may be expressed to increase short chain (8:0-10:0) fatty acids (Dehesh et al. (1998) Plant J. 15:383-390). Additionally, thioesterases may be overexpressed to lower intracellular concentrations of fatty acyl-ACPs and acyl-CoA to release feedback inhibition on the fatty acid biosynthesis pathway (Lu et al., ibid)

A complementary strategy to increase activity of fatty acid biosynthesis pathways is to decrease fatty acid and lipid catabolism. This may be accomplished by diminishing degradation of fatty acids through β-oxidation. It is well understood that if there are several compartments with β-oxidation activity, as is frequently the case in eukaryotes with e.g. β-oxidation occurring in the peroxisome and in the mitochondrion, β-oxidation is ideally diminished in all these compartments.

The first step in fatty acid catabolism of a free fatty acid is frequently activation by an acyl-CoA synthetase to an acyl-CoA molecule. Long chain acyl-CoA synthetases are described by the EC numbers 6.2.1.3 and 6.2.1.10, short chain acyl-CoA synthetases by the EC number 6.2.1.2. It is preferable to delete acyl-CoA synthetase activity for fatty acids with chain lengths of more than two carbon atoms in the fatty acid producing biocatalyst. For example in E. coli, long chain acyl-CoA synthetase is encoded by fadD, and deletion of the gene abolished β-oxidation of long chain fatty acids (Kunau et al. (1995) Prog. Lipid Res. 34:267-342).

Other central enzymes in the β-oxidation of long chain fatty acids are acyl-CoA dehydrogenase (or acyl-CoA oxidase, usually replacing acyl-CoA dehydrogenase in the peroxisomal β-oxidation cycle), enoyl-CoA hydratase, β-hydroxyl-CoA dehydrogenase and acyl-CoA acetyltransferase (thiolase). Some of these enzyme activities are also found in the biosynthesis pathway. However, deletion of one or more of these enzyme activities that are active in degradation may lead to an improved production and/or accumulation of fatty acids and/or lipids.

Another favorable way to achieve high fatty acid and/or lipid formation is by reducing flux to other metabolites and/or storage compounds. This is achieved through producing fatty acids and/or lipids in a medium with substrates that provide elements for production of lipids, e.g. carbon, oxygen and hydrogen, whereas the medium is limiting or even deprived of substrates providing elements for production of alternative metabolites. For example nitrogen-containing substrates such as ammonium, urea or amino acids are required for the production of amino acids and nucleic acids, or phosphorus providing substrates such as phosphate are required for the synthesis of DNA and RNA or polyphosphate storage products. Other limiting or deprived components in a media favorable for fatty acid and/or lipid production can be vitamins or metal ions that are utilized by enzymes of pathways competing with lipid biosynthesis, as e.g. iron, zinc or magnesium (Beopoulos et al. (2009) Prog Lipid Rex. 48:375-387).

Another favorable approach to increase the cellular lipid content is down-regulating metabolic pathways that lead to the production of metabolites and storage compounds other than fatty acids and lipids by means of diminishing their biosynthesis pathways. One prominent class of non-lipid storage compounds are carbohydrates, e.g. starch, trehalose and others. Competing biosynthesis pathways may be down-regulated or deleted. For example, interrupting the starch biosynthesis pathway by e.g. deletion of ADP glucose phosphorylase leads to a significant increase of free fatty acids as well as triacylglycerols in the algae Chlamydomonas reinhardtii (Wang et al. (2009) Eukaryot. Cell. 8:1856-1868).

Strains of E. coli that have been genetically engineered to produce increased amounts of fatty acids where the fatty acids accumulate as free fatty acids are described in Lu et al. ((2008) Metabolic Eng. 10:333-339). The engineering included knocking out the endogenous fadD gene encoding an acyl-CoA synthetase to block fatty acid degradation, expressing a plant thioesterase to increase the abundance of shorter chain fatty acids, overexpressing acetyl-CoA carboxylase (ACC) to increase the supply of malonyl-CoA, and overexpressing an endogenouse thioesterase to release feedback inhibition caused by long chain fatty acyl-acyl carrier proteins (ACP).

A strain of E. coli that was engineered to overproduce medium-chain fatty acids (mostly C12 and C14) is described in Lennen et al. ((2010) Biotech. and Bioeng. 106:193-202). The engineering included eliminating beta-oxidation by deletion of the fadD gene encoding acetyl-CoA synthetase, the first enzyme involved in beta-oxidation, overexpressing the four subunits of acetyl-CoA carboxylase, and expressing a plant acyl-acyl carrier protein thioesterase from Umbellularia californica. Increased fatty acid production was also obtained in E. coli by engineering cells for cytosolic expression of E. coli thioesterase, which is naturally localized to the periplasm (Steen et al. (2010) Nature 463:559-562). The described or other strains of E. coli, or of other microorganisms, that are engineered as described above to produce increased amounts of fatty acids, may be used in the present process.

Oleaginous yeast is a preferred microbe for fatty acid production, as these microorganisms can commonly accumulate in excess of about 25% of their dry cell weight as oil. Examples of oleaginous yeast include, but are not limited to, the following genera: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, Lipomyces. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica).

For example, the oleaginous yeast Yarrowia lipolytica has been genetically engineered to produce high amounts of the fatty acid eicosapentaenoic acid (cis-5, 8, 11, 14, 17-eicosapentaenoic acid; ω-3) as disclosed in U.S. Pat. Appl. Pub. No. 2006-0115881-A1. Some references describing means to engineer the oleaginous host organism Yarrowia lipolytica for EPA biosynthesis are provided as follows: U.S. Pat. No. 7,238,482, U.S. Pat. No. 7,550,286, U.S. Pat. Appl. Pub. No. 2006-0115881-A1, U.S. Pat. Appl. Pub. No. 2009-0093543-A1, and U.S. Pat Appl. Pub. No. 2010-0317072, which are hereby incorporated herein by reference. For example, optimized recombinant Yarrowia lipolytica strains for EPA production with demonstrated production of up to 55.6% EPA of total fatty acids in a recombinant Y. lipolytica strain were obtained by engineering for expression of the following genes: Δ9 elongase, Δ8 desaturase, Δ5 desaturase, Δ17 desaturase, Δ12 desaturase, C_{16/18} elongase and diacylglycerol cholinephosphotransferase, in a host cell comprising a disruption in the native peroxisome biogenesis factor 10 protein (PEX10). Examples of suitable Y. lipolytica strains that may be used in the present process include, but are not limited to, Y. lipolytica strains designated as ATCC #20362, ATCC #8862, ATCC #18944, ATCC #76982, Y4305, Y8672, Y8647, and LGAM S(7)1 (Papanikolaou S., and Aggelis G., Bioresour. Technol. 82(1):43-9 (2002)).

In addition to production of fatty acids or alcohol by separate microorganisms which are natural producers or are genetically engineered for production of these products, a microorganism may be genetically engineered for production of both fatty acids and alcohols using pathways and genetic engineering methods described above.

Fatty acids produced by a microorganism may be in the form of free fatty acids, but are more typically fatty acid derivatives (typically esters) such as in the form of triacylglycerols (TAGs), diacylglycerols, and monoacyglycerols that are components of oils and waxes. In addition, fatty acids derivatives may be saccharolipids and phospholipids. For purposes herein microbial waxes containing fatty acids will be considered to be a type of microbial oil. Microbial oils, which may also be called lipids, are typically stored intracellularly, and may also be released into the medium during fermentation. To render microbially produced fatty acids accessible for esterification with a microbially produced alcohol as in the present method, fatty acids and/or oil products are released from the microbial production cell either naturally or by applied methods. Release may be by methods including physical methods, chemical methods, biological methods (including natural methods such as natural cell lysis), enzymatic methods, and/or genetic methods that result in extracellular secretion of the fatty acid and/or oil products.

Mechanical forces that are typically applied for disrupting cells result from impingement, friction, tension, pressure, extrusion, cavitation, sonication, or electroporation. Generation of these forces for cell disruption is well known to one of skill in the art. The devices that make use of these forces usually exert several of these forces for maximum efficiency. Particularly suited for use to release fatty acids are sonication, shock waves (US 2008/0311638), bead beating/ball milling/grinding, or high-pressure homogenizers (WO/2008/019964, US 2005/0197409).

Additional chemical/physical methods for disrupting cells to release fatty acids and/or oil include generation of acidic or caustic conditions by acids or bases, respectively, using solvents to dissolve or disrupt the cell membranes, and thermal/physical methods such as high temperatures, freeze/thaw cycles, high or low osmotic pressure. Also cells may be disrupted by the use of chemicals such as addition of anionic detergents, which may be followed by protease digestion.

To facilitate release of fatty acids and/or oil, also biological means can be applied. Apoptosis occurs in most living systems, including microalgae, fungi, and bacteria. Apoptosis is an energy-dependent process in which dying cells undergo controlled autolysis. Consequently, addition of compounds that induce apoptosis, such as acriflavine in yeast (Leyhani et al. (2009) Ann. NY Acad. Sci. 1171:2840291), represent a method to cause autolysis and release fatty acids and oil into the cell broth.

Cells with cell walls can be made more sensitive to lysis procedures by applying enzymes or mixtures thereof that weaken cell walls. Examples of such enzyme activities are lysozyme, chitinase and lysostaphin.

Lysozymes, also known as muramidase or N-acetylmuramide glycanhydrolase, are glycoside hydrolases, of the enzyme class EC 3.2.1.17, that damage bacterial cell walls by catalyzing hydrolysis of 1,4-beta-linkages between N-acetylmuramic acid and N-acetyl-D-glucosamine residues in a peptidoglycan and between N-acetyl-D-glucosamine residues in chitodextrins. Chitinases (EC 3.2.1.14) are digestive enzymes that break down glycosidic bonds in chitin and bear the systematic name (1->4)-2-acetamido-2-deoxy-beta-D-glucan glycanohydrolase, also some chitinase enzymes also display lysozyme (EC 3.2.1.17) activity. Lysostaphin (EC 3.4.24.75), e.g. from Staphylococcus simulans, is a metalloendopeptidase. Enzymes such as these may used as autolytic enzymes and may be introduced with regulated expression to trigger autolysis of cells releasing fatty acids and/or oil.

Examples of mixtures of enzyme activites are lyticase, consisting of at least the two synergistic enzyme activities of a β-1,3-glucanase and an alkaline protease as described for Oerskovia xanthineolytica (Scott and Schekman (1980) J. Bacteriol. 142:414-423). The lytic activity of zymolase, e.g. purified from Arthrobacter luteus, consists of several synergistic enzyme activities as well, in particular of Zymolyase A, beta-1,3-glucan laminaripentaohydrolase and Zymolyase B as well as alkaline protease (AMS Biotechnology, Abingdon, UK). Glusulase is a preparation of the intestinal juice of the snail Helix pomatia. It is a deep brown, slightly viscous (gel-free) liquid. It contains a mixture of enzymes including β-glucuronidase, sulfatase, and a cellulose (Perkin Elmer, Waltham, Mass.).

Strains of a microorganism that secrete fatty acids or TAGs into culture broth may be identified by mutagenesis and screening as described by Nojima et al. ((1999) J. Gen. Appl. Microbiol. 45:1-6). Elevating intracellular fatty acid concentrations can increase fatty acid secretion, which may be accomplished through the down-regulation and/or deletion of acyl-CoA synthetases. If no acyl-CoA synthetases are available for “activating” fatty acids by thioester formation, excess fatty acids are released into the supernatant, as described by Scharnewski et al. after e.g. deletion of acyl-CoA synthetases FAA1 and FAA4 in S. cerevisiae (Scharnewski et al. (2008) Febs J. 275:2765-2778). A similar form of fatty acid secretion is described by Roessler et al. for cyanobacteria (WO/2009/076559).

Alternatively, mechanisms for lipid transport or secretion of plant cells, such as ATP binding cassette ABCG12/CER5 or ABCG11/WBC11, described to transport waxes in Arabidopsis thaliana (Pighin et al. (2004) Science 306:702-704; Bird et al. (2007) Plant J. 52:485-498), may be used in cells of microorganisms. Homologous proteins identified by sequence analysis, hybridization techniques or mutation analysis, may be optimized by means of protein engineering to efficiently transport desired product fatty acids, and subsequently over-expressed in a microbial production host.

Another way to permeabilize and/or lyse cells in order to release fatty acids and/or oils is by the use of autolytic strains. Autolysis of cells may be implemented as a continuous process in a fraction of the cell population or is induced at a desired time point in the process through the depletion or addition of a trigger molecule in the culture broth, like e.g. oxygen, an amino acid, etc. One method of initiating autolysis of a microorganism is by down-regulating enzymes involved in the biogenesis of the cell wall. In yeast, autolysis in biofuel production has been achieved by down-regulating activities of SRB1p/PSA1p and PKC1p (Zhang et al. (1999) Biotechnol Bioeng 64:607-615). Autolysis may also be induced as an autophagy response, which has been correlated in yeast with expression of Csc1-1, whose expression was shown to accelerate autolysis in an industrial strain for fermentation of grape juice to wine (Cebollero et al. (2009) Tiotechnol. Prog. 25:1598-16040.

Permeabilization of membranes and lysis of cells can also be achieved with the controlled expression or down-regulation of regulatory proteins, such as cell lysis in yeast by expression of GAL4 (Martegani et al. (1993) Yeast 9:575-582) or by down-regulation of BCY1 (Tabera et al. (2006) Appl. Environ. Microbiol. 72; 2351-2358).

Also, proteases can be expressed in cells for inducing cell lysis. For example, HIV-1 protease arrests yeast growth and finally results in cell lysis. The lytic phenotype includes loss of plasma membrane integrity and cell wall breakage leading to the release of cell contents to the medium (Elanco et al. (2003) J. Biol. Chem. 278:1086-1093). Alternatively proteases with comparable proteinase activity as well as regulatory features can be identified in screening systems and by means of bioinformatics and can be expressed in biocatalysts for biodiesel production.

The means for releasing fatty acids and/or oils are either applied continuously during the production process, or in selected phases of the production process, e.g. at the end of the process. They are either applied to the whole fermentation broth, or to a selected fraction of it. A process may be run continuously, with a portion of the fermentation broth periodically removed for fatty acid release. Before applying the means for releasing lipids as aforementioned, cells may optionally be concentrated (e.g., to at least about 100 g dry cell weight/L or more, including to at least about 120 g dry cell weight/1, 150 g dry cell weight/1, 175 g dry cell weight/L, 200 g dry cell weight/L or more). At least one, or any combination of the aforementioned regimes may be used.

When microbially produced fatty acids are present as derivatives such as monoacylglycerols, diacylglycerols, triacylglycerols, or mixtures thereof, a catalyst is used to hydrolyze the derivatives into free fatty acids or to directly transesterify the triacylglycerols with alcohol as described below. In addition, saccharolipids and phospholipids may be hydrolyzed by a catalyst. Fatty acids used in the present process may be of any carbon chain length that is at least C₆, and are typically in the range of C₆ to C₂₂ in carbon chain length. A fatty acid alcohol ester having a minimum total carbon chain length of C₁₀ is typically desired as biodiesel fuel. Thus a four carbon butanol esterified with a C₆ fatty acid is desirable, or a longer chain fatty acid may be esterified with an alcohol of fewer carbons. The fatty acids may be saturated or unsaturated, though preferred fatty acids for diesel fuel are the saturated lauric acid (12:0) and myristic acid (14:0). Two or more different fatty acids may be present in microbially produced fatty acids used for esterification.

In the present process microbially produced alcohol is combined with microbially produced fatty acids and/or oil and contacted with a catalyst in a fermentation medium. The catalyst is capable of esterifying free fatty acids with alcohol to form fatty acid alkyl esters extracellularly. In one embodiment the catalyst can transesterify triacylglycerols to fatty acid alcohol esters. Optionally the catalyst is capable of hydrolyzing fatty acid derivatives, such as the triacylglycerols of oils (including waxes), into free fatty acids. In one embodiment a second catalyst may be added to hydrolyze fatty acid derivatives producing free fatty acids for esterification.

The glycerol product of acylglycerol hydrolysis may be used as a carbon source by the microorganism producing alcohol and/or fatty acid products. Typically glycerol would be a supplemental carbon source, in addition to sugars in the fermentation medium.

In some embodiments, the catalyst and the optional second catalyst, can be one or more enzymes, for example carboxylic ester hydrolyases (EC 3.1.1). In some embodiments, the catalyst can be one or more enzymes, for example, hydrolase enzymes such as lipase enzymes. Lipases are carboxylic ester hydrolyases that catalyze the hydrolysis of long-chain acylglycerols, but which generally also show activity for hydrolysis of a broad range of ester substrates. The active site of a lipase consists of three catalytic residues: a nucleophilic serine residue, a catalytic acid residue (aspartate or glutamate), and a histidine residue, always in this order in the amino acid sequence of the lipase. The nucleophilic serine residue is located in a highly conserved Gly-X-Ser-X-Gly pentapeptide.

Lipase enzymes used in the present process may be derived from any source, including, for example, Absidia, Achromobacter, Aeromonas, Alcaligenes, Alternaria, Aspergillus, Achromobacter, Aureobasidium, Bacillus, Beauveria, Brochothrix, Candida, Chromobacter, Coprinus, Fusarium, Geotricum, Hansenula, Humicola, Hyphozyma, Lactobacillus, Metarhizium, Mucor, Nectria, Neurospora, Paecilomyces, Penicillium, Pseudomonas, Rhizoctonia, Rhizomucor, Rhizopus, Rhodosporidium, Rhodotorula, Saccharomyces, Sus, Sporobolomyces, Thermomyces, Thiarosporella, Trichoderma, Verticillium, and/or a strain of Yarrowia. In a preferred aspect, the source of the lipase is selected from the group consisting of Absidia blakesleena, Absidia corymbifera, Achromobacter iophagus, Alcaligenes sp., Alternaria brassiciola, Aspergillus flavus, Aspergillus niger, Aureobasidium pullulans, Bacillus pumilus, Bacillus strearothermophilus, Bacillus subtilis, Brochothrix thermosohata, Candida cylindracea (Candida rugosa), Candida paralipolytica, Candida Antarctica lipase A, Candida antartica lipase B, Candida ernobii, Candida deformans, Chromobacter viscosum, Coprinus cinerius, Fusarium oxysporum, Fusarium solani, Fusarium solani pisi, Fusarium roseum culmorum, Geotricum penicillaturn, Hansenula anomala, Humicola brevispora, Humicola brevis var. thermoidea, Humicola insolens, Lactobacillus curvatus, Rhizopus oryzae, Penicillium cyclopium, Penicillium crustosum, Penicillium expansum, Penicillium sp. I, Penicillium sp. II, Pseudomonas aeruginosa, Pseudomonas alcaligenes, Pseudomonas cepacia (syn. Burkholderia cepacia), Pseudomonas fluorescens, Pseudomonas fragi, Pseudomonas maltophilia, Pseudomonas mendocina, Pseudomonas mephitica lipolytica, Pseudomonas alcaligenes, Pseudomonas plantari, Pseudomonas pseudoalcaligenes, Pseudomonas putida, Pseudomonas stutzeri, and Pseudomonas wisconsinensis, Rhizoctonia solani, Rhizomucor miehei, Rhizopus japonicus, Rhizopus microsporus, Rhizopus nodosus, Rhodosporidium toruloides, Rhodotorula glutinis, Saccharomyces cerevisiae, Sporobolomyces shibatanus, Sus scrofa, Thermomyces lanuginosus (formerly Humicola lanuginose), Thiarosporella phaseolina, Trichoderma harzianum, Trichoderma reesei, and Yarrowia lipolytica. In a further preferred aspect, the lipase is selected from the group consisting of Thermomcyces lanuginosus, Aspergillus sp. lipase, Aspergillus niger lipase, Candida antartica lipase B, Pseudomonas sp. lipase, Penicillium roqueforti lipase, Penicillium camembertii lipase, Mucor javanicus lipase, Burkholderia cepacia lipase, Alcaligenes sp. lipase, Candida rugosa lipase, Candida parapsilosis lipase, Candida deformans lipase, lipases A and B from Geotrichum candidum, Neurospora crassa lipase, Nectria haematococca lipase, Fusarium heterosporum lipase Rhizopus delemar lipase, Rhizomucor miehei lipase, Rhizopus arrhizus lipase, and Rhizopus oryzae lipase. Suitable commercial lipase preparations suitable as enzyme catalyst include, but are not limited to Lipolase® 100 L, Lipex® 100L, Lipoclean® 2000T, Lipozyme® CALB L, Novozym® CALA L, and Palatase 20000L, available from Novozymes (Bagsvaerd, Denmark), or from Pseudomonas fluorescens, Pseudomonas cepacia, Mucor miehei, hog pancreas, Candida cylindracea, Candida rugosa, Rhizopus niveus, Candida antarctica, Rhizopus arrhizus or Aspergillus available from SigmaAldrich.

The catalyst is present in a fermentation medium also containing at least one microorganism that produces alcohol, fatty acid and/or oil, or both alcohol and fatty acid and/or oil products. The catalyst may be added before, after, or contemporaneously with fatty acids to an alcohol containing fermentation medium. Alternatively, the catalyst may be added before, after, or contemporaneously with alcohol to a fatty acid/oil containing fermentation medium. The catalyst may be a single enzyme or a mixture of enzymes. In another aspect the catalyst may be expressed by either the microorganism producing the alcohol or the fatty acid and either secreted into the medium or alternatively released into the medium through cell lysis. In addition, the medium contains at least one carbon source, at least one alcohol, and at least one free fatty acid. The fatty acids produced by a microorganism may be in the form of triacylglycerol (TAG), diacylglycerol (DAG), monoacylglycerol (MAG), or mixtures thereof in an oil as described previously. Free fatty acids are derived from microbial oil by hydrolysis of TAG, DAG, and/or MAG in the oil, which may occur at any time prior to esterification. Fatty acid alcohol esters are produced by action of the esterifying catalyst in situ, in the fermentation medium.

In addition to the fatty acids and/or oil produced by a microorganism, the fermentation medium may contain additional fatty acids and oil that may be used in forming fatty acid alcohol esters. For example, the use of grain, such as corn, as a feedstock for liquefaction to provide sugars in the fermentation medium will result in an oil component in the resulting mash.

In one embodiment, a microorganism producing alcohol is grown photosynthetically or in a fermentation medium, and fatty acids and/or oil produced by a second microorganism are added to the photosynthetic culture medium or fermentation medium, which also contains the catalyst described above, and the alcohol product. The fatty acids and/or oil may be produced by the second microorganism in a separate fermentation, or photosynthetically. The fatty acids and/or oil may be added as a component of the fermentation medium of the second microorganism, or of the photosynthetic culture of the second microorganism, or may be partially purified or purified prior to addition. Partial purification or purification may be, for example, by hexane extraction. Oil may by hydrolyzed to release free fatty acids prior to or following addition to the fermentation medium containing alcohol. Hydrolysis is typically catalyzed either by the esterifying catalyst, or by a second catalyst as described above. Fatty acid alcohol esters (fatty acid alkyl esters) are produced by action of the esterifying catalyst (for example, lipase) in situ, in the fermentation medium of the alcohol-producing microorganism.

In another embodiment, a microorganism producing fatty acid(s) or oil is grown photosynthetically or in a fermentation medium, and alcohol produced by a second microorganism in a separate photosynthetic culture medium or fermentation medium is added to the culture or fermentation medium, which also contains the catalyst described above, and the fatty acid or oil product of fermentation. The fatty acids and/or oil are released using a method as described above. The alcohol may be added as a component of the fermentation medium of the second microorganism, or of the photosynthetic culture of the second microorganism, or may be partially purified or purified prior to addition. Partial purification or purification may be, for example, by distillation. Fatty acid alcohol esters are produced by action of the esterifying catalyst in situ, in the fermentation medium of the fatty acid-producing microorganism.

Fermentative production of fatty acids at high titer levels can be challenging since many (but not all) long chain fatty acids are solids at temperatures typically used in fermentation processes. The presence of solids during fermentation creates challenges for adequate mixing that negatively affects efficient oxygen transfer and homogeneous mixing in the vessel, and may additionally make the separation of the fermentation broth from a solid phase more difficult or costly than the separation of a fermentation broth from a second liquid organic phase prior to product recovery. This problem may be overcome by feeding alcohol to the fermentation vessel containing a microorganism that secretes a fatty acid product into the medium in the presence of the esterifying enzyme, producing a fatty acid alcohol ester that is a liquid at the temperature of fermentation. Since the fed alcohol and the secreted fatty acid are esterified in situ immediately forming a second phase, the concentration of fatty acid in the vessel can be maintained below the threshold level at which it may individually form a solid phase. The alcohol can be added to the fermentation medium at a concentration that is not significantly inhibitory or toxic to the microorganism producing fatty acid(s) and/or oil, where the alcohol partitions between the fermentation broth and the second organic phase comprised of fatty acid alcohol ester, fatty acid, oil and alcohol. Where both the fatty acid and the fatty acid alcohol ester are individually solids at the fermentation temperature, the production of a mixture of said fatty acid and fatty acid alcohol ester may form a mixture (potentially also comprising alcohol and/or oil) having a melting temperature below the fermentation temperature, again preventing the formation of a solid phase during the fermentation.

In some embodiments, the concentration of fatty acid, or oil comprising fatty acid and/or a mixture of triglycerides, diglycerides and monglycerides, in the culture or fermentation medium is sufficient to form a two-phase mixture comprising an aqueous phase and an organic phase. Under these conditions, where lipase catalyzes the reversible reaction of fatty acid (or glyceride) and alcohol to produce fatty acid alcohol ester and water (or partially or completely hydrolyzed glyceride), product fatty acid alcohol esters partition predominately into the organic phase, reducing their concentration in the aqueous phase and thereby driving the conversion of fatty acid (or glycerides) to fatty acid alcohol esters by continuously removing them from the aqueous phase at the aqueous/organic interface where the lipase is active. This state of generating a two-phase aqueous/organic mixture during fermentation is desired to drive the esterification reaction to high conversion.

In another embodiment the same microorganism produces an alcohol and fatty acids and/or oil as it grows photosynthetically or in a fermentation medium containing at least carbon source. A catalyst in the fermentation medium esterifies the alcohol and fatty acids extracellularly.

In yet another embodiment, a microorganism producing alcohol and a microorganism producing fatty acids and/or oil are grown in the same fermentation medium. The co-cultured microorganisms may utilize one or more of the same fermentable sugars as carbon substrate. Alternatively, each microorganism utilizes a different fermentable sugar from the same pool of fermentable sugars. For example, one microorganism may produce ethanol utilizing xylose, and another microorganism may produce fatty acids utilizing glucose, where both xylose and glucose are present in the fermentation medium.

Through the combination of microbially-produced alcohol and fatty acid, in the present process different alcohols and fatty acids may be esterified as desired. Fuel properties of biodiesel such as cetane number, heat of combustion, propensity for cold flow, oxidative stability, viscosity, lubricity, and composition of exhaust emissions are affected by the fatty acid and alcohol components. For example, structural features of incorporated fatty acids including chain length, degree of unsaturation, and branching of the carbon chain affect properties of fatty acid alcohol esters. The structure of alcohol also affects fuel properties. For example, one major draw back of fatty acid methyl esters (FAME) as biodiesel is its poor properties at low temperatures indicated by high cloud point and pour point. Cold temperature fuel properties of biodiesel can be improved by use of branched esters such as iso-propyl, iso-butyl, and 2-butyl esters.

The fatty acid alcohol esters produced in situ in the present process are obtained from the fermentation broth using any method of separating fatty acid alcohol esters from the other components of fermentation broth. For example, the fatty acid alcohol esters may form one phase of a biphasic mixture with fermentation broth, allowing separation of the phase containing the fatty acid alcohol esters from an aqueous fermentation broth phase. Separation of this biphasic mixture into ester-containing organic phase and aqueous phase can be achieved using any methods known in the art, including but not limited to, siphoning, aspiration, decantation, centrifugation, using a gravity settler, membrane-assisted phase splitting, and the like. All or part of the resulting aqueous phase can be recycled into the fermentation vessel as fermentation medium, or otherwise discarded and replaced with fresh medium, or treated for the removal of any remaining product alcohol or fatty acid and then recycled to fermentation vessel.

### EXAMPLES

As used herein, the meaning of abbreviations used was as follows: “μg” means microgram(s), “g” means gram(s), “kg” means kilogram(s), “L” means liter(s), “mL” means milliliter(s), “mL/L” means milliliter(s) per liter, “mL/min” means milliliter(s) per min, “DI” means deionized, “uM” means micrometer(s), “nM” means nanometer(s), “w/v” means weight/volume, “OD” means optical density, “OD₆₀₀” means optical density at a wavelength of 600 nM, “dcw” means dry cell weight, “rpm” means revolutions per minute, “° C.” means degree(s) Celsius, “° C./min” means degrees Celsius per minute, “slpm” means standard liter(s) per minute, “ppm” means part per million, “pdc” means pyruvate decarboxylase enzyme followed by the enzyme number, “h” means hour(s), “min” means minute(s), FABE means fatty acid butyl ester(s), “FAEE” means fatty acid ethyl ester(s), “PUFA” means polyunsaturated fatty acid(s), “TFA” means total fatty acids, “DCW” means dry cell weight.

### General Methods

Samples analyzed for fatty acid butyl esters are run on an Agilent 6890 GC with a 7683B injector and a G2614A auto sampler. The column is an HP-DB-FFAP column (15 meters×0.53 mm ID (Megabore), 1-micron film thickness column (30 m×0.32 mm ID, 0.25 μm film). The carrier gas is helium at a flow rate of 3.7 mL/min measured at 45° C. with constant head pressure; injector split is 1:50 at 225° C.; oven temperature is 100° C. for 2.0 min, 100° C. to 250° C. at 10° C./min, then 250° C. for 9 min for a run time of 26 minutes. Flame ionization detection is used at 300° C. with 40 mL/min helium makeup gas. Standards (Nu-Chek Prep; Elysian, Minn.) are used to confirm the identity of fatty acid alcohol ester products.

### Yarrowia Strains

The construction of the oleaginous yeast Yarrowia lipolytica strains Y8647 and Y8672 is described in US2010/0317072, which is incorporated herein by reference. Example 4 therein describes the construction of strain Y8647 which was derived from Yarrowia lipolytica ATCC #20362. This strain is capable of producing about 53.6% EPA relative to total lipids with 37.6% total lipid content [“TFAs % DOW”] via expression of a A9 elongase/A8 desaturase pathway. The lipid profile is given in Table 3. Fatty acid nomenclature is given in Table 4.

The final genotype of strain Y8647 with respect to wildtype Yarrowia lipolytica ATCC #20362 is Ura+, Pex3−, unknown 1−, unknown 2−, unknown 3−, unknown 4−, unknown 5−, unknown6−, unknown 7−, unknown 8−, YAT1::ME3S::Pex16, GPD::ME3S::Pex20, YAT1::ME3S::Lip1, FBAlNm::EgD9eS::Lip2, EXP1::EgD9eS::Lip1, GPAT::EgD9e::Lip2, YAT1::EgD9eS::Lip2, FBAlNm::EgD8M::Pex20, EXP1::EgD8M::Pex16, FBAlN::EgD8M::Lip1, GPD::EaD8S::Pex16, YAT1::E389D9eS/EgD8M::Lip1, FBAlNm::EaD9eS/EaD8S::Lip2, GPD::FmD12::Pex20, YAT1::FmD12::Oct, EXP1::FmD12S::Aco, GPDIN::FmD12::Pex16, EXP1::EgD5M::Pex16, FBAlN::EgD5SM:Pex20, GPDIN::EgD5SM::Aco, GPM::EgD5SM::Oct, YAT1::EaD5SM::Oct, FBAlNm::PaD17::Aco, EXP1::PaD17::Pex16, YAT1::PaD17S::Lip1, YAT1::YICPT::Aco, YAT1::MCS::Lip1.

The structure of the above expression cassettes are represented by a simple notation system of “X::Y::Z”, wherein X describes the promoter fragment, Y describes the gene fragment, and Z describes the terminator fragment, which are all operably linked to one another. Abbreviations are as follows: FmD12 is a Fusarium moniliforme delta-12 desaturase gene [U.S. Pat. No. 7,504,259]; FmD12S is a codon-optimized delta-12 desaturase gene, derived from F. moniliforme [U.S. Pat. No. 7,504,259]; ME3S is a codon-optimized C_{16/18} elongase gene, derived from Mortierella alpina [U.S. Pat. No. 7,470,532]; EgD9e is a Euglena gracilis delta-9 elongase gene [U.S. Pat. No. 7,645,604]; EgD9eS is a codon-optimized delta-9 elongase gene, derived from E. gracilis [U.S. Pat. No. 7,645,604]; EgD8M is a synthetic mutant delta-8 desaturase gene [U.S. Pat. No. 7,709,239], derived from E. gracilis [U.S. Pat. No. 7,256,033]; EaD8S is a codon-optimized delta-8 desaturase gene, derived from Euglena anabaena [U.S. Pat. No. 7,790,156]; E389D9eS/EgD8M is a DGLA synthase created by linking a codon-optimized delta-9 elongase gene (“E389D9eS”), derived from Eutreptiella sp. CCMP389 delta-9 elongase (U.S. Pat. No. 7,645,604) to the delta-8 desaturase “EgD8M” (supra) [U.S. Pat. Appl. Pub. No. 2008-0254191-A1]; EaD9eS/EgD8M is a DGLA synthase created by linking a codon-optimized delta-9 elongase gene (“EaD9eS”), derived from E. anabaena delta-9 elongase (U.S. Pat. 7,794,701) to the delta-8 desaturase “EgD8M” (supra) [U.S. Pat. Appl. Pub. No. 2008-0254191-A1]; EgD5M and EgD5SM are synthetic mutant delta-5 desaturase genes [U.S. Pat. Appl. Pub. No. 2010-0075386-A1], derived from Euglena gracilis [U.S. Pat. No. 7,678,560]; EaD5SM is a synthetic mutant delta-5 desaturase gene [U.S. Pat. Appl. Pub. No. 2010-0075386-A1], derived from Euglena anabaena [U.S. Pat. No. 7,943,365]; PaD17 is a Pythium aphanidermatum delta-17 desaturase gene [U.S. Pat. No. 7,556,949]; PaD17S is a codon-optimized delta-17 desaturase gene, derived from P. aphanidermatum [U.S. Pat. No. 7,556,949]; YICPT1 is a Yarrowia lipolytica diacylglycerol cholinephosphotransferase gene [U.S. Pat. No. 7,932,077]; and, MCS is a codon-optimized malonyl-CoA synthetase gene, derived from Rhizobium leguminosarum bv. viciae 3841 [U.S. Pat. Appl. Pub. No. 2010-0159558-A1].

Example 7 of US2010/0317072 describes the construction of strain Y8672 which was derived from Yarrowia lipolytica ATCC #20362. This strain is capable of producing about 61.8% EPA relative to the total lipids with 26.5% total lipid content [“TFAs % DOW”] via expression of a Δ9 elongase/Δ8 desaturase pathway. The lipid profile is given in Table 3. The final genotype of strain Y8672 with respect to wildtype Yarrowia lipolytica ATCC #20362 is Ura+, Pex3−, unknown 1−, unknown 2−, unknown 3−, unknown 4−, unknown 5−, unknown 6−, unknown 7−, unknown 8−, Leu+, Lys+, YAT1::ME3S::Pex16, GPD::ME3S::Pex20, GPD::FmD12::Pex20, YAT1::FmD12::Oct, EXP1::FmD12S::ACO, GPAT::EgD9e::Lip2, FBAlNm::EgD9eS::Lip2, EXP1::EgD9eS::Lip1, YAT1::EgD9eS::Lip2, FBAlNm::EgD8M::Pex20, FBAlN::EgD8M::Lip1, EXP1::EgD8M::Pex16, GPD::EaD8S::Pex16 (2 copies), YAT1::E389D9eS/EgD8M::Lip1, YAT1::EgD9eS/EgD8M::Aco, FBAlN::EgD5SM::Pex20, YAT1::EgD5SM::Aco, GPM::EgD5SM::Oct, EXP1::EgD5M::Pex16, EXP1::EgD5SM::Lip1, YAT1::EaD5SM::Oct, YAT1::PaD17S::Lip1, EXP1::PaD17::Pex16, FBAlNm::PaD17::Aco, GPD::YICPT1::Aco, YAT1::MCS::Lip1.

EgD9ES/EgD8M is an abbreviation for a DGLA synthase created by linking the delta-9 elongase “EgD9eS” (supra) to the delta-8 desaturase “EgD8M” (supra) [U.S. Pat. Appl. Pub. No. 2008-0254191-A1].

TABLE 3
Total Lipid Content And Composition (as % of total)
In Yarrowia Strains Y8647 and Y8672 in flask assay

| Strain | 16:0 | 16:1 | 18:0 | 18:1 | 18:2 | ALA | EDA | DGLA | ARA |
|---|---|---|---|---|---|---|---|---|---|
| Y8647 | 1.3 | 0.2 | 2.1 | 4.7 | 20.3 | 1.7 | 3.3 | 3.6 | 0.7 |
| Y8672 | 2.3 | 0.4 | 2.0 | 4.0 | 16.1 | 1.4 | 1.8 | 1.6 | 0.7 |

| | | | | | DCW | TFAs % | EPA % |
|---|---|---|---|---|---|---|---|
| Strain | EtrA | ETA | EPA | other | (g/L) | DCW | DCW |
| Y8647 | 0.6 | 3.0 | 53.6 | 4.5 | 3.8 | 37.6 | 20.1 |
| Y8672 | 0.4 | 1.1 | 61.8 | 6.4 | 3.3 | 26.5 | 16.4 |

TABLE 4
Fatty acid nomenclature

| | | | Shorthand |
|---|---|---|---|
| Common Name | Abbreviation | Chemical Name | Notation |
| Palmitic | Palmitate | hexadecanoic | 16:0 |
| Palmitoleic | — | 9-hexadecenoic | 16:1 |
| Stearic | — | octadecanoic | 18:0 |
| Oleic | — | cis-9-octadecenoic | 18:1 |
| Linoleic | LA | cis-9,12-octadecadienoic | 18:2 ω-6 |
| α-Linolenic | ALA | cis-9,12,15- | 18:3 ω-3 |
| | | octadecatrienoic | |
| Eicosadienoic | EDA | cis-11,14-eicosadienoic | 20:2 ω-6 |
| Dihomo-γ- | DGLA | cis-8,11,14-eicosatrienoic | 20:3 ω-6 |
| Linolenic | | | |
| Arachidonic | ARA | cis-5,8,11,14- | 20:4 ω-6 |
| | | eicosatetraenoic | |
| Eicosatrienoic | ETrA | cis-11,14,17-eicosatrienoic | 20:3 ω-3 |
| Eicosa- | ETA | cis-8,11,14,17- | 20:4 ω-3 |
| tetraenoic | | eicosatetraenoic | |
| Eicosa- | EPA | cis-5,8,11,14,17- | 20:5 ω-3 |
| pentaenoic | | eicosapentaenoic | |

### Example 1

### Prophetic

### Production of Fatty Acids by Euglena Gracilis

Euglena gracilis is cultivated in a Braun B plus 1 L fermenter (Sartorius, Goettingen, Germany). The cultivation is performed with a defined medium as follows: 10 g/L glucose, 0.8 g/L KH₂PO₄, 1.5 g/L (NH₄)₂SO₄, 0.5 g/L MgSO₄.7H₂O, 0.2 g/L CaCO₃, 0.0144 g/L H₃BO₃, 2.5 mg/L vitamin B₁, 20 mg/L vitamin B₁₂, 1 ml/L trace elements solution, and 1 ml/L Fe solution. The trace elements solution contains 4.44 g/L ZnSO₄.7H₂O, 0.12 g/L MnSO4.H₂O, 0.03 g/L Na₂MoO₄.2H₂O, 0.03 g/L CuSO₄.5H₂O and 0.04 g/L CoSO₄.5H₂O in distilled water. The Fe solution contains 0.11 g/L (NH₄)₂SO₄Fe(SO₄)₂.6H₂O and 0.1 g/L EDTA in distilled water. Growth conditions are 28° C., pH=2.8 and a gas flow of 1 vvm nitrogen to achieve anaerobic conditions. The outside of the fermenter is covered with aluminium foil to grow the culture in the dark. The culture produces fatty acids under anaerobic conditions through a malonyl-independent fatty acid biosynthesis pathway using acetyl-CoA and propionyl-CoA as C2- and C3-donors.

### Example 2

### Prophetic

### Production of Fatty Acids by Rhodococcus Opacus

Rhodococcus opacusis cultivated in a Braun B plus 1 L fermenter (Sartorius, Goettingen, Germany). The cultivation is performed in batch mode with a defined medium containing 240 g/L glucose, 13.45 g/L (NH₄)₂SO₄, 1 g/L MgSO₄.7H₂O, 0.015 g/L CaCl₂.7H₂O, 1 mL/L trace element solution, 1 mL/L solution 1, and 35.2 mL/L 1M phosphate buffer. C/N ratio is 17.8. The trace element solution contains (per L of water): FeS0₄.7H₂O: 0.5 g, ZnSO.7H₂O: 0.4 g, MnS0₄.H₂O: 0.02 g, H₃BO₃: 0.015 g, NiC1₂.6H₂O: 0.01 g, EDTA: 0.25 g, CoCl₂.6H₂O: 0.05 g, and CuCl₂.2H₂O: 0.005 g. Solution 1 contains (per L of water) NaMoO₄.2H₂O: 2.0 g and FeNa.EDTA: 5.0 g. Inoculum OD660 is about 1.0. Growth conditions are 30° C., pH=6.9. PH control is accomplished by addition of 1M NaOH. Dissolved oxygen is maintained above 30% oxygen saturation, air gas flow at 0.5 vvm by controlling stirrer speed in the range of 400-2000 rpm. For foam control polypropylene glycol P2000 (Fluka) is used. High intracellular triacyl glycerol accumulation is observed.

### Example 3

### Prophetic′

### Production of Fatty Acids by Botryococcus Braunii

Botryococcus braunii is cultivated in a Braun B plus 1 L fermenter (Sartorius, Goettingen, Germany). The medium is modified Chu 13 medium, containing per L of de-ionized water: KNO₃: 400 mg, K₂HPO₄: 80 mg, CaCl₂.2H₂O: 107 mg, MgSO₄.7H₂O: 200 mg, Ferric Citrate: 20 mg, Citric acid: 100 mg, CoCl₂: 0.02 mg, H₃BO₃: 5.72 mg, MnCl2.4H₂O: 3.62 mg, ZnSO₄.7H₂O: 0.44 mg, CuSO₄.5H₂O 0.16 mg, Na₂MoO₄: 0.084 mg, 0.072N H₂SO₄: 1 drop. The fermenter is inoculated with 20% (v/v) of a two-week-old pre-culture. Temperature is 26° C., stirrer speed is 300 rpm, the glass vessel is exposed to 1.2 klux light intensity in a 16:8 h light:dark cycle, aeration is 0.1 vvm with water-saturated air or water-saturated air enriched with CO₂. Cultivation is carried out for 25 days. Significant lipid production is observed. Biomass and fatty acid containing lipid content is increased in cultures that are cultivated in an atmosphere enriched with CO₂.

### Example 4

### Prophetic′

### Production of Fatty Acids by Mortierella isabellina

Mortierella isabellina is cultivated in a Braun B plus 1 L fermenter (Sartorius, Goettingen, Germany). Fermentation media contains glucose: 100 g/L, (NH₄)₂SO₄: 0.5 g/L, Yeast Extract: 0.5 g/L, KH₂PO₄: 7 g/L, Na₂HPO₄: 2 g/L, MgSO₄.7H₂O: 1.5 g/L, CaCl₂.2H₂O: 0.1 g/L, FeCl₃.6H₂O: 0.008 g/L, ZnSO₄.7H₂O: 0.001 g/L, CuSO₄.5H₂O: 0.0001 g/L, Co(NO₃)₂.H₂O: 0.0001 g/L, MnSO₄.5H₂O: 0.0001 g/L. Growth conditions are 28° C., pH is controlled at pH=6.0. Dissolved oxygen is maintained above 30% oxygen saturation, air gas flow at 0.5 vvm by controlling stirrer speed in the range of 400-2000 rpm. High fatty acid containing lipid accumulation is observed.

### Example 5

### Prophetic′

### Production of Fatty Acids by Yarrowia lipolytica

Yarrowia lipolytica is cultivated in a Braun B plus 1 L fermenter (Sartorius, Goettingen, Germany). Fermentation media contains glycerol: 30 g/L, Yeast Extract: 0.5 g/L, (NH₄)₂SO₄: ad molar C/N ratio of C/N=100, KH₂PO₄: 7 g/L, Na₂HPO₄: 2.5 g/L, MgSO₄.7H₂O: 1.5 g/L, CaCl₂.2H₂O: 0.15 g/L, FeCl₃.6H₂O: 0.15 g/L, ZnSO₄.7H₂O: 0.02 g/L, MnSO₄.5H₂O: 0.06 g/L. Growth conditions are 28° C., pH is controlled at pH=6.0 with 2 M KOH. Dissolved oxygen is maintained above 30% oxygen saturation, air gas flow at 1.0 vvm by controlling stirrer speed in the range of 400-2000 rpm. Fatty acid containing lipid accumulation is observed.

### Example 6

### Prophetic′

Production of Fatty Acids by Lipomyces starkeyi

Lipomyces starkeyi is cultivated in a Braun B plus 1 L fermenter (Sartorius, Goettingen, Germany). Fermentation media contains (NH₄)₂SO₄: 3 g/L, KH₂PO₄: 20 g/L, MgSO₄.7H₂O: 2 g/L, CaCl₂.2H₂O: 0.1 g/L, Fe: 10 mg/L, Zn: 10 mg/L, Mn: 10 mg/L, Cu: 0.5 mg/L, Na₂MoO₄.2 H₂O: 10 mg/L, CoCl₂.6H₂O: 10 mg/L, KI: 1 mg/L, H₃BO₃: 1 mg/L, biotin: 0.2 μg/L. Growth conditions are 30° C., pH is controlled at pH=4.0 with 10N NaOH. Dissolved oxygen is maintained at 2.5 ppm by controlling stirrer speed in the range of 400-2000 rpm with a manually adjusted air gas flow in the range of 0.1-1.0 vvm. Ethanol is fed in a fed-batch mode and ethanol concentration in the fermenter is controlled at 2.5 g/L. To prevent foaming, 20% Silicone KM-72 is used as antifoam solution. Lipid formation is triggered with depletion of either nitrogen, Zn or Fe available. To achieve either nitrogen, Zn or Fe limitations, initial concentrations of nitrogen, Zn or Fe are reduced to support an OD of 5 at 570 nm in the bioreactor cultivations, respectively. Intracellular fatty acid containing lipid accumulation is observed.

### Example 7

### Prophetic′

Production of Fatty Acids by Chlorella Protothecoides

Chlorella protothecoides is cultivated in a Braun B plus 1 L fermenter (Sartorius, Goettingen, Germany). The composition of the culture medium is as follows: Glucose: 20 g/L, Yeast Extract: 4 g/L, KH₂PO₄: 0.7 g/L, K₂HPO₄: 0.3 g/L, MgSO₄.7H₂O: 0.3 g/L, FeSO₄.7H₂O: 3 mg/L, glycine: 0.1 g/L, vitamin B1: 0.01 mg/L, A5 trace mineral solution: 1 ml/L. A5 trace mineral solution comprises H₃BO₃: 2.86 g/L, Na₂MoO₄.2H₂O: 0.039 g/L, ZnSO₄.7H₂O: 0.222 g/L, MnCl₂.4H₂O: 1.81 g/L, CuSO₄.5H₂O: 0.074 g/L. Chlorella growth factor mix containing 0.5-4% protein, 1-3% sugar, 1% free amino acids, and 0.01% plant hormones is added at 0.1% (v/v). Glucose concentration in the fermenter is controlled in the range of 10 g/L to 24 g/L. Growth temperature is 28° C., pH is controlled at pH=6.0 with 1M KOH. Dissolved oxygen is maintained above 40% by controlling stirrer speed in the range of 400-2000 rpm with a manually adjusted air gas flow of 1.0 vvm. High intracellular fatty acid containing lipid accumulation is detected.

### Example 8

### Prophetic′

### Production of Fatty Acids by Rhodosporidium Toruloides

Rhodosporidium toruloides Y4 is cultivated in a in a Braun B plus 1 L fermenter (Sartorius, Goettingen, Germany). Initial media volume including seed culture is 800 mL. The culture medium contains: glucose: 60 g/L, peptone: 15 g/L, Yeast Extract: 15 g/L. Glucose concentration is maintained above 20 g/L, by feeding sterilized glucose powder in 30 g portions. Growth conditions are 30° C., pH is controlled at pH=5.6 with 10 M NaOH. Dissolved oxygen is maintained at 40% oxygen saturation with an air gas flow at 1 vvm by controlling stirrer speed in the range of 400-2000 rpm. Initial cDW is 1 g/L. Fatty acid containing lipids and free fatty acids accumulate in the cells.

### Example 9

### Prophetic′

### Production of Fatty Acid Ethyl Alcohol Ester by Lipase-Catalyzed Reaction of Ethanol and Microbial Fatty Acids During Fermentation of Yeast

The wild-type yeast strain CEN.PK113-7D is propagated overnight in medium containing yeast nitrogen base without amino acids (6.7 g/L), dextrose (25 g/L) and MES buffer (0.1 M at pH 5.5). The overnight culture is diluted into fresh medium such that the resulting optical density at 600 nm is 0.1. The diluted culture is aliquoted, 25 mL per flask, into six 250 mL sealed-cap shake flasks. Four of the cultures are supplemented with either of two lipase enzyme stock solutions (2 mg protein/mL 10 mM phosphate buffer (pH 7.0) of Lipozyme® CALB L or Lipolase® 100L) to a final lipase concentration of 10 ppm in the media. Microbial oil containing fatty acids is added at a 1:1 volume ratio to the aqueous culture in three of the flasks (no enzyme, CALB L, or Lipolase 100L). One flask has no supplements. The cultures are grown in a temperature-controlled shaking incubator at 30° C. and a shaking speed of 250 rpm for 23 hours. Samples (5 mL aqueous or 10 mL culture/fatty acid emulsion) for chromatographic analysis are immediately centrifuged for 5 minutes at 4000 rpm in a TX-400 swinging bucket rotor in 15 mL conical bottom tubes. For aqueous samples, a 0.22 μm spin filter is used prior to analysis. Aqueous samples are analyzed on a Shodex SH1011 column with a SH-G guard column using 0.01M sulfuric acid mobile phase at 50° C. and a flow rate of 0.5 mL per minute. Detection of sugars and alcohols is by Refractive Index and 210 nm absorption, and quantitation is performed using standard curves. Samples are taken of the aqueous culture (no added microbial fatty acid) or culture/fatty acid emulsion, and analyzed as described in General Methods for ethyl esters of fatty acids. Fatty acid methyl esters are produced in fermentations containing lipase and microbial oil.

### Example 10

### Prophetic′

### Production of 1-Propyl-Fatty Acid Ester by Lipase-Catalyzed Reaction of 1-Propanol and Microbial Oil Fatty Acids

Reaction mixtures containing aqueous 2-(N-morpholino)ethanesulfonic acid buffer (0.20 M, pH 5.5), 1-propanol, lipase (Lipolase® 100 L (Novozymes), Lipozyme® CALB L (Novozymes), Rhizopus arrhizus lipase (SigmaAldrich), and Candida cylindracea lipase (SigmaAldrich) and microbial oil fatty acids are stirred at 30° C. Samples are withdrawn while stirring from each reaction mixture at predetermined times, immediately centrifuged, and the aqueous and organic layers are separated and analyzed for 1-propanol and 1-propyl esters of microbial oil fatty acids. These esters are produced.

### Example 11

### Prophetic′

### In Situ Microbial Fatty Acid Esterification with Butanol Produced by Engineered Yeast Using Lipase

### Seed Flask Growth

A Saccharomyces cerevisiae strain that was engineered to produce isobutanol from a carbohydrate source, with pdc1 deleted, pdc5 deleted, and pdc6 deleted is grown from a stock culture stored at −80° C. to 0.55-1.1 g/L dcw (OD₆₀₀1.3-2.6—Thermo Helios α Thermo Fisher Scientific Inc., Waltham, Mass.) in seed flasks from a frozen culture. The culture is grown at 26° C. in an incubator rotating at 300 rpm. The composition of the first seed flask medium is:
3.0 g/L dextrose
3.0 g/L ethanol, anhydrous
3.7 g/L ForMedium Synthetic Complete Amino Acid (Kaiser) Drop-Out: -HIS, -URA (Reference # DSCK162CK)
6.7 g/L Difco Yeast Nitrogen Base without amino acids (#291920)

Twelve milliliters from the first seed flask culture is transferred to a 2 L flask and grown at 30° C. in an incubator rotating at 300 rpm. The second seed flask has 220 mL of the following medium:
30.0 g/L dextrose
5.0 g/L ethanol, anhydrous
3.7 g/L ForMedium Synthetic Complete Amino Acid (Kaiser) Drop-Out:
   without HIS, without URA (Reference # DSCK162CK) 6.7 g/L Difco Yeast Nitrogen Base without amino acids (#291920) 0.2M MES Buffer titrated to pH 5.5-6.0

The culture is grown to 0.55-1.1 g/L dcw (OD₆₀₀ 1.3-2.6). An addition of 30 mL of a solution containing 200 g/L peptone and 100 g/L yeast extract is added at this cell concentration. Then an addition of 300 mL of 0.2 uM filter sterilized Cognis, 90-95% oleyl alcohol is added to the flask. The culture continues to grow to >4 g/L dcw (OD₆₀₀>10) before being harvested and added to the fermentation.

### Fermentation Preparation

### Initial Fermentation Vessel Preparation

A glass jacked, 2 L fermentation vessel (Sartorius A G, Goettingen, Germany) is charged with house water to 66% of the liquefaction weight. A pH probe (Hamilton Easyferm Plus K₈, part number: 238627, Hamilton Bonaduz A G, Bonaduz, Switzerland) is calibrated through the Sartorius DCU-3 Control Tower Calibration menu. The zero is calibrated at pH=7. The span is calibrated at pH=4. The probe is then placed into the fermentation vessel, through the stainless steel head plate. A dissolved oxygen probe (pO₂ probe) is also placed into the fermentation vessel through the head plate. Tubing used for delivering nutrients, seed culture, extracting solvent, and base are attached to the head plate and the ends are foiled. The entire fermentation vessel is placed into a Steris (Steris Corporation, Mentor, Ohio) autoclave and sterilized in a liquid cycle for 30 minutes.

The fermentation vessel is removed from the autoclave and placed on a load cell. The jacket water supply and return line is connected to the house water and clean drain, respectively. The condenser cooling water in and water out lines are connected to a 6-L recirculating temperature bath running at 7° C. The vent line that transfers the gas from the fermentation vessel is connected to a transfer line that is connected to a Thermo mass spectrometer (Prima dB, Thermo Fisher Scientific Inc., Waltham, Mass.). The sparger line is connected to the gas supply line. The tubing for adding nutrients, extract solvent, seed culture, and base is plumbed through pumps or clamped closed.

The fermentation vessel temperature is controlled at 55° C. with a thermocouple and house water circulation loop. Wet corn kernels (#2 yellow dent) are ground using a hammer mill with a 1.0 mm screen, and the resulting ground whole corn kernels are then added to the fermentation vessel at a charge that is 29-30% (dry corn solids weight) of the liquefaction reaction mass.

### Liquefaction

An alpha amylase is added to the fermentation vessel per its specification sheet while the fermentation vessel is mixing at 300-1200 rpm, with sterile, house N₂ being added at 0.3 slpm through the sparger. The temperature set-point is changed from 55° C. to 85° C. When the temperature is >80° C., the liquefaction cook time is started and the liquefaction cycle is held at >80° C. for 90-120 minutes. The fermentation vessel temperature set-point is set to the fermentation temperature of 30° C. after the liquefaction cycle is complete. N₂ is redirected from the sparger to the head space to prevent foaming without the addition of a chemical antifoaming agent.

### Nutrient Addition Prior to Inoculation

Add 6.36 mL/L (post-inoculation volume) of ethanol (200 proof, anhydrous) just prior to inoculation. Add thiamine to 20 mg/L final concentration just prior to inoculation. Add 100 mg/L nicotinic acid just prior to inoculation.

Fermentation vessel Inoculation

The fermentation vessel pO₂ probe is calibrated to zero while N₂ is being added to the fermentation vessel. The fermentation vessels pO₂ probe is calibrated to its span with sterile air sparging at 300 rpm. The fermentation vessel is inoculated after the second seed flask is >4 g/L dcw. The inoculum is pumped into the fermentation vessel through a peristaltic pump.

Microbial oil is added at a 1:1 volume ratio to the aqueous medium in the fermentation vessel. Lipase enzyme stock (2 mg protein/mL 10 mM phosphate buffer (pH 7.0) of Lipozyme® CALB L or Lipolase® 100L) is added to a final lipase concentration of 10 ppm.

### Fermentation Vessel Operating Conditions

The fermentation vessel is operated at 30° C. for the entire growth and production stages. The pH is allowed to drop from a pH between 5.7-5.9 to a control set-point of 5.2 without adding any acid. The pH is controlled for the remainder of the growth and production stage at a pH=5.2 with ammonium hydroxide. Sterile air is added to the fermentation vessel, through the sparger, at 0.3 slpm for the remainder of the growth and production stages. The pO₂ is set to be controlled at 3.0% by the Sartorius DCU-3 Control Box PID control loop, using stir control only, with the stirrer minimum being set to 300 rpm and the maximum being set to 2000 rpm. The glucose is supplied through simultaneous saccharification and fermentation of the liquified corn mash by adding α-amylase (glucoamylase). The glucose is kept in excess (1-50 g/L) for as long as starch is available for saccharification.

Samples are taken of the culture/fatty acid emulsion, and analyzed as described in General Methods for ethyl esters of fatty acids. Fatty acid butyl esters are produced in the fermentation containing lipase and microbial oil.

### Example 12

### Production of Fatty Acid Isobutyl Alcohol Esters by Lipase-Catalyzed Reaction Of Isobutanol with Fatty Acids Produced by Yarrowia lipolytica Using Cell Lysate

Yarrowia lipolytica strain ATCC 20362 was cultivated in a 125-mL shake flask. Glycerol stock of the strain was inoculated in 3 mL YPD medium (Yeast Extract (10 g/L), Bacto Peptone (20 g/L), D-glucose (20 g/L)) and incubated with shaking at 200 rpm for 24 hours at 30° C. The resulting culture was diluted to an initial OD₆₀₀ of 0.3 in 10 mL of D-FM medium (urea (1 g/L), Bacto YE (5 g/L), KH₂PO₄ (6 g/L), Na₂HPO₄.12H₂O (3.2 g/L), D-glucose (40 g/L)) and incubated with shaking at 200 rpm for 24 hours at 30° C. Glucose buffer (10 mL; K₂HPO₄ (12.6 g/L), KH₂PO₄ (3.8 g/L), NaHCO₃ (16.8 g/L) and D-glucose (320 g/L)) were added directly to the flask and the flask was incubated with shaking at 200 rpm for 120 hours at 30° C. (final cell concentration of 35 OD₆₀₀).

A 2-mL aliquot of the resulting fermentation broth was mixed with 0.8 g of acid-washed glass beads by vortexing at 4000 rpm for 15 min to produce a cell lysate (12 mg dry cell weight/mL). Cell lysate (0.446 mL) was mixed with 0.050 mL of an aqueous solution containing 100 ppm Candida antarctica lipase B (Lipozyme® CALB L (Novozymes); Sigma/Aldrich catalog number L3170) and isobutanol (4 μL of a 0.8 g/mL isobutanol solution in water, 6.4 mg/mL isobutanol final concentration) and incubated at 30° C. for 8 h with shaking at 250 rpm in a Thermomixer (Eppendorf) for esterification. Control reactions were also run that omitted the addition of isobutanol and/or lipase to the reaction mixture (Table 5). Product fatty acid isobutyl alcohol esters (FABE) were extracted into 0.2 mL hexane and analyzed by GC and GC-MS using an Agilent DB-FFAP column (30 meters×0.25 mm ID, 0.25 micron film thickness). FABE products were quantified using standards (Nu-Chek Prep; Elysian, Minn.). GC/MS of reaction samples identified C16:0 FABE, C16:1 FABE, C18:0 FABE, C18:1 FABE and C18:2 FABE as major reaction products in the reaction containing isobutanol; total FABE content was 62 mg/L in reaction containing 10 ppm lipase, compared to 54 mg/mL total FABE in a control reaction containing isobutanol but no added lipase. No FABE was produced in control reactions lacking added isobutanol.

TABLE 5
FABE content. Data are averages of two independent
experiments.

| | | | | | Total |
|---|---|---|---|---|---|
| | | | | Sum of FABE | FABE |
| Experiment | Lysate | Isobutanol | Lipase | areas | (mg/L) |
| C1, control | + | + | − | 1210986 | 54 |
| C2, control | + | − | + | 0 | 0 |
| C3, control | + | − | − | 0 | 0 |
| Exp 1 | + | + | + | 1376837 | 62 |

### Example 13

### Production of Fatty Acid Ethyl Alcohol Esters by Lipase-Catalyzed Reaction of Ethanol with Fatty Acids Produced by Yarrowia lipolytica

Yarrowia lipolytica strain ATCC 20362 was cultivated in a 125-mL shake flask. Glycerol stock of the strain was inoculated in 3 mL YPD medium (Yeast Extract (10 g/L), Bacto Peptone (20 g/L), D-glucose (20 g/L)) and incubated with shaking at 200 rpm for 24 hours at 30° C. The resulting culture was diluted to an initial OD₆₀₀ of 0.3 in 10 mL of D-FM medium (urea (1 g/L), Bacto YE (5 g/L), KH₂PO₄ (6 g/L), Na₂HPO₄.12H₂O (3.2 g/L), D-glucose (40 g/L)) and incubated with shaking at 200 rpm for 24 hours at 30° C. Glucose buffer (10 mL; K₂HPO₄ (12.6 g/L), KH₂PO₄ (3.8 g/L), NaHCO₃ (16.8 g/L) and D-glucose (320 g/L)) were added directly to the flask and the flask was incubated with shaking at 200 rpm for 120 hours at 30° C. (final cell concentration of 35 OD₆₀₀).

A 2-mL aliquot of the resulting fermentation broth was mixed with 0.8 g of acid-washed glass beads by vortexing at 4000 rpm for 15 min to produce a cell lysate (12 mg dry cell weight/mL). Cell lysate (0.446 mL) was mixed with 0.050 mL of an aqueous solution containing 100 ppm Candida antarctica lipase B (Lipozyme® CALB L (Novozymes); Sigma/Aldrich catalog number L3170) and ethanol (4 μL of a 0.8 g/mL ethanol solution in water, 6.4 mg/mL ethanol final concentration) at 30° C. for 8 h with shaking at 250 rpm in a Thermomixer (Eppendorf) for esterification. Product fatty acid ethyl alcohol esters (FAEE) were extracted into 0.2 mL hexane and analyzed by GC and GC-MS as described in Example 12. GC/MS identified C16:0 FAEE, C16:1 FAEE, C18:0 FAEE, C18:1 FAEE and C18:2 FAEE as major reaction products in the reaction containing ethanol. The percentages of the total FAEE for individual FAEE components is listed in Table 6.

TABLE 6
FAEE peak areas by GC-MS.

| | Retention Time | | % total |
|---|---|---|---|
| Compounds | (min) | Area | FAEE |
| FAEE C16:0 | 17.43 | 299884 | 25.86 |
| FAEE C16:1 | 17.682 | 111097 | 9.58 |
| FAEE C18:0 | 19.202 | 184192 | 15.89 |
| FAEE C18:1 | 19.383 | 473845 | 40.87 |
| FAEE C18:2 | 19.784 | 90411.5 | 7.80 |
| total | | 1159430 | |

### Example 14

### Production of Eicosapentaenoic Acid Isobutyl Alcohol Ester by Lipase-Catalyzed Reaction of Isobutanol with Eicosapentaenoic Acid Produced by Yarrowia lipolytica

Inocula of Yarrowia lipolytica strains Y8672 and Y8647 (described in General Methods) were prepared from frozen cultures in a shake flask. After an incubation period, the culture was used to inoculate a fermentor. The fermentation was a 2-stage fed-batch process. In the first stage, the yeast was cultured under conditions that promoted rapid growth to a high cell density; the culture medium comprised glucose, various nitrogen sources, trace metals and vitamins. In the second stage, the yeast were starved for nitrogen and continuously fed glucose to promote lipid and polyunsaturated fatty acid (PUFA) accumulation. Process variables including temperature (controlled between 30-32° C.), pH (controlled between 5-7), dissolved oxygen concentration and glucose concentration were monitored and controlled per standard operating conditions to ensure consistent process performance and final PUFA oil quality.

Aliquots (1.6 mL) of the resulting fermentation broth containing either Yarrowia lipolytica strain Y8672 or strain Y8647 were mixed with glass beads by vortexing in 2-mL microcentrifuge tubes for 3×3 min to produce a mechanically-prepared cell lysate. The cell lysates prepared in multiple tubes containing fermentation broth of the same strain were combined. Reaction mixtures containing 13.83 g of Yarrowia lipolytica strain Y8672 or strain Y8647 fermentation broth either with or without mechanical cell lysis treatment, were mixed with 1.08 g of isobutanol and 0.188 mL of an aqueous solution containing 2 mg protein/mL of Thermomyces lanuginosus lipase in 10 mM potassium phosphate buffer (Lipolase® 100 L (Novozymes); Sigma/Aldrich catalog number L0777) and incubated at 30° C. with magnetic stirring at 300 rpm. The concentration of lipase in the reaction mixture was 25 ppm, based on BCA protein analysis (Thermo Scientific, Rockford, Ill.). Control reactions were run that omitted the addition of lipase to the reaction mixture.

Reaction mixture samples (0.50 g) were mixed with 4.5 mL of isopropanol containing methyl pentadecanoate as internal standard, and the resulting mixtures centrifuged and the supernatants analyzed for FABE, including the isobutyl ester of eicosapentaenoic acid, by GC using an Agilent DB-FFAP column (30 meters×0.32 mm ID, 1.0 micron film thickness): iBu Palmitate, 15.550 min; iBu Stearate, 17.100 min; iBu Oleate, 17.260 min; iBu Linoleate, 17.750 min; iBu Linolenate, 18.300 min; iBu Eicosanoate, 20.250 min. FABE products were quantified using standards (Nu-Chek Prep; Elysian, Minn.). Isobutyl ester of eicosapentaenoic acid (EPA) was prepared as follows: a 100 μL aliquot of heptane containing 1-1.5 mg of EPA (Nu Chek), was placed into a 100×13 mm pyrex tube, then 1 ml of ˜10% (v:v) acetyl chloride in anhydrous isobutanol was added, the tube tightly capped with a Teflon™-lined screw cap and heated at 80° C. for 1 h. After cooling to room temperature, 1 mL of 1M NaCl and 400 μL heptane was added, the tube, vortexed, and after the phases partitioned, ˜300 μL of the organic phase was transferred to a GC vial prior to analysis. GC-MS was performed to confirm identity of reaction components using an J&W DB-Wax column (30 meters×0.32 mm ID, 0.5 micron film thickness). GC and GC/MS analysis of reaction samples confirmed that the isobutyl ester of eicosapentaenoic acid was the major reaction product in reactions containing isobutanol. Concentrations of the isobutyl ester of eicosapentaenoic acid and total FABE is reported in Table 7.

TABLE 7
Isobutyl ester of eicosapentaenoic acid production using high
EPA-producing Yarrowia strains

| | mechanical | Time | Lipase | iBu Eicosanoate | total FABE |
|---|---|---|---|---|---|
| Strain | lysis | (h) | (ppm) | (mg/L) | (mg/L) |
| Y8672 | + | 0 | 25 | 205 | 308 |
| Y8672 | + | 24 | 25 | 922 | 1383 |
| Y8672 | + | 72 | 25 | 493 | 739 |
| Y8672 | + | 0 | 0 | 0 | 0 |
| Y8672 | + | 24 | 0 | 0 | 0 |
| Y8672 | + | 72 | 0 | 0 | 0 |
| Y8672 | − | 0 | 25 | 0 | 0 |
| Y8672 | − | 24 | 25 | 537 | 927 |
| Y8672 | − | 72 | 25 | 659 | 1368 |
| Y8672 | − | 0 | 0 | 0 | 0 |
| Y8672 | − | 24 | 0 | 0 | 0 |
| Y8672 | − | 72 | 0 | 0 | 0 |
| Y8647 | + | 0 | 25 | 216 | 486 |
| Y8647 | + | 24 | 25 | 150 | 600 |
| Y8647 | + | 72 | 25 | 524 | 892 |
| Y8647 | + | 0 | 0 | 0 | 154 |
| Y8647 | + | 24 | 0 | 0 | 103 |
| Y8647 | + | 72 | 0 | 0 | 102 |
| Y8647 | − | 0 | 25 | 0 | 142 |
| Y8647 | − | 24 | 25 | 88 | 353 |
| Y8647 | − | 72 | 25 | 138 | 505 |
| Y8647 | − | 0 | 0 | 0 | 93 |
| Y8647 | − | 24 | 0 | 0 | 87 |
| Y8647 | − | 72 | 0 | 0 | 140 |

What is claimed is:

1. A process for the production of fatty acid alcohol esters comprising:
a) providing a microorganism which produces at least one alcohol;
b) providing a microorganism which produces at least one free fatty acid or a microbial oil comprising at least one triacylglycerol, diacylglycerol, or monoacylglycerol, or mixtures thereof;
c) growing the microorganism of a) in a medium comprising:
i) optionally at least one fermentable carbon source;
ii) a catalyst capable of esterifying free fatty acids with alcohol into fatty acid alkyl esters and optionally capable of hydrolyzing acylglycerols into free fatty acids; and
iii) at least one free fatty acid derived from the microorganism of b);
wherein fatty acid alcohol esters are formed extracellularly and in situ from esterification of the free fatty acids with the alcohol using the catalyst.

2. A process for the production of fatty acid alcohol esters comprising:
a) providing a microorganism which produces at least one alcohol;
b) providing a microorganism which produces at least one fatty acid or a microbial oil comprising at least one triacylglycerol, diacylglycerol, or monoacylglycerol, or mixtures thereof;
c) growing the microorganism of b) in a medium comprising:
i) optionally at least one fermentable sugar;
ii) a catalyst capable of esterifying free fatty acids with alcohol into fatty acid alkyl esters and optionally capable of hydrolyzing acylglycerols into free fatty acids; and
iii) at least one alcohol derived from the microorganism of a);
wherein fatty acid alcohol esters are formed extracellularly and in situ from esterification of the free fatty acids with the alcohol using the catalyst.

3. The process of either of claim 1 or 2 wherein the fermentable carbon source is derived from biomass.

4. The process of claim 1 or 2 wherein the at least one fatty acid or a microbial oil is a liquid in the medium.

5. The process of claim 1 or 2 wherein the fatty acid alcohol esters are a liquid in the medium.

6. The process of claim 1 or 2 wherein the at least one fatty acid or a microbial oil is a solid in the medium.

7. The process of claim 1 or 2 wherein the fatty acid alcohol esters are a solid in the medium.

8. The process of claim 1 or 2 wherein the microorganisms of step a) and b) are independently selected from the group consisting of a yeast, a bacteria, a cyanobacteria, a protist, a microalgae, and a filamentous fungus.

9. The process of claim 8 wherein the microorganisms of step a) and b) are independently a photosynthetic microorganism.

10. The process of claim 8 wherein the microorganism of step a) or b) is a microorganism independently selected from the group consisting of: Zygosaccharomyces, Schizosaccharomyces, Kluyveromyces, Yarrowia, Dekkera, Torulopsis, Brettanomyces, Pichia, Candida, Hansenula Saccharomyces, Issatchenkia, Clostridium, Zymomonas, Escherichia, Salmonella, Serratia, Erwinia, Shigella, Rhodococcus, Pseudomonas, Streptomyces, Bacillus, Lactobacillus, Enterococcus, Alcaligenes, Klebsiella, Paenibacillus, Arthrobacter, Corynebacterium, Brevibacterium, Euglena, Botryococcus, Mortierella, Lipomyces, Chlorella, Rhodosporidium, Trochosporon, Thraustochytrids, Chlamydomonas, Dunaliella, Hematococcus, Scenedesmus, Synechocystis, Synechococcus Arhtospira, Cyclotella, Hantzschia, Nitzschia, and Ankistrodesmus.

11. The process of claim 1 or 2 wherein the at least one free fatty acid is derived from the microbial oil.

12. The process of claim 1 or 2 wherein the at least one fatty acid is the end product of a metabolic pathway in a microorganism.

13. The process of claim 1 or 2 wherein the microorganism of step a) is the same as the microorganism of step b).

14. The process of claim 1 or 2 wherein the microorganism of step a) and the microorganism of step b) are independently selected from the group consisting of an anaerobe, a facultative anaerobe, and an aerobe.

15. The process of claim 1 or 2 wherein the alcohol produced by the microorganism of a) is a C1 to C8 straight chain or branched chain alcohol.

16. The process of claim 1 or 2 wherein the at least one free fatty acid is a saturated or unsaturated C6-C22 free fatty acid.

17. The process of claim 1 or 2 wherein the at least one free fatty acid is produced via a malonyl-CoA dependent biosynthesis pathway.

18. The process of claim 1 or 2 wherein the at least one free fatty acid is produced via a malonyl-CoA independent biosynthesis pathway.

19. The process of claim 1 or 2 wherein the microorganism of a) produces the at least one alcohol by way of an endogenous metabolic pathway.

20. The process of claim 1 or 2 wherein the microorganism of a) produces the at least one alcohol by way of a recombinant metabolic pathway.

21. The process of claim 20 wherein the recombinant metabolic pathway comprises at least one gene that is not native to the wildtype of the microorganism.

22. The process of claim 1 or 2 wherein the microorganism of step b) is oleaginous.

23. The process of claim 22 wherein the oleaginous microorganism is capable of producing oil at a concentration of at least about 20% of its cellular dry weight.

24. The process of claim 23 wherein the oleaginous microorganism is a yeast selected from the group consisting of Yarrowia, Candida, Rhodotorula, Rhodosporidium, Cryptococcus, Saccharomyces, Hanselula, Pichia, Aspergillis Mortierella, Conidiobolus, Pythium, Phytophathora, Penicillium, Porphyridium, Coidosporium, Mucor, Fusarium, Entomophthora, Trichosporon and Lipomyces or an algae selected from the group consisting of Scenedesmus, Chlorella, Cyclotella, Hantzschia, Nitzschia, and Ankistrodesmus.

25. The process of claim 1 or 2 wherein the microorganism of step b) secretes fatty acids or a microbial oil.

26. The process of claim 1 or 2 wherein the microorganism of step b) releases the microbial oil by autolysis.

27. The process of claim 26 wherein the microorganism of step b) comprises an autolytic enzyme.

28. The process of claim 1 or 2 wherein the oil is released from the microorganism of step b) by mechanical disruption.

29. The process of claim 1 or 2 wherein the microorganisms of step a) and step b) are independently capable of producing the catalyst of step c) ii).

30. The process of claim 1 or 2 wherein the fermentable carbon source is selected from the group consisting of monosaccharides, disaccharides, oligosaccharides, glycerol, and mixtures thereof.

31. The process of claim 1 or 2 wherein the microorganisms of step a) and step b) are grown in the same medium utilizing one or more of the same fermentable carbon sources for growth.

32. The process of claim 1 or 2 wherein the microorganisms of step a) and step b) are grown in the same fermentation vessel each utilizing a different fermentable carbon source using the same pool of fermentable carbon sources.

33. The process of claim 1 or 2 wherein the microorganisms of step a) and step b) are grown in separate media.

34. The process of claim 1 or 2 wherein the catalyst capable of esterifying free fatty acids with alcohol into fatty acid alkyl esters is an enzyme.

35. The process of claim 34 wherein the enzyme is a lipase.

36. The process of claim 35 wherein the lipase is derived from a microorganism selected from the group consisting of Absidia, Achromobacter, Aeromonas, Alcaligenes, Alternaria, Aspergillus, Achromobacter, Aureobasidium, Bacillus, Beauveria, Brochothrix, Candida, Chromobacter, Coprinus, Fusarium, Geotricum, Hansenula, Humicola, Hyphozyma, Lactobacillus, Metarhizium, Mucor, Nectria, Neurospora, Paecilomyces, Penicillium, Pseudomonas, Rhizoctonia, Rhizomucor, Rhizopus, Rhodosporidium, Rhodotorula, Saccharomyces, Sus, Sporobolomyces, Thermomyces, Thiarosporella, Trichoderma, Verticillium, and Yarrowia.

37. The process of claim 1 or 2 wherein the free fatty acids are formed from hydrolysis of at least a portion of the at least one triacylglycerol, diacylglycerol, or monoacylglycerol in the oil using the catalyst.

38. The process of claim 1 or 2 wherein the microorganism of step b) produces eicosapentaenoic acid and eicosapentaenoic acid-alcohol esters are produced.

39. An alcohol fermentation process composition comprising:
a) a processed biomass comprising water and fermentable carbon source;
b) a catalyst capable of esterifying free fatty acids with alcohol into fatty acid alkyl esters and optionally capable of hydrolyzing acylglycerols into free fatty acids;
c) at least one alcohol produced by a microorganism;
d) free fatty acids produced by a microorganism;
e) optionally a microbial oil comprising at least one triacylglycerol, diacylglycerol, or monoacylglycerol, or mixtures thereof; and
f) fatty acid alcohol esters formed in situ from esterification of the free fatty acids with the alcohol using the catalyst.

40. The composition of claim 39 wherein the processed biomass is selected from the group consisting of processed corn grain, wheat, rye, barley, rice, corn cobs, crop residues such as corn husks, corn stover, grasses, wheat straw, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

41. The composition of claim 39 wherein the alcohol is a C1 to C8 straight chain or branched chain alcohol.

41. The composition of claim 39 wherein the catalyst is an enzyme.

42. The composition of claim 39 wherein the oil is produced by an oleaginous microorganism
